(19) United States
(12) Patent Application Publication
(10) Pub. No.: US 2022/0370631 A1
(43) Pub. Date: Nov. 24, 2022

(54) COMBINATION THERAPY OF CARBOPLATIN AND NAPI2B-TARGETED POLYMER ANTIBODY-DRUG CONJUGATE FOR THE TREATMENT OF OVARIAN CANCER

(71) Applicant: Mersana Therapeutics, Inc., Cambridge, MA (US)

(72) Inventor: Robert A. BURGER, Jenkintown, PA (US)

(21) Appl. No.: 17/735,575

(22) Filed: May 3, 2022

Related U.S. Application Data
(60) Provisional application No. 63/242,323, filed on Sep. 9, 2021.
(60) Provisional application No. 63/183,420, filed on May 3, 2021.

(51) Int. Cl. A61K 47/68; A61K 31/282; A61P 35/00; C07K 16/32; A61P 15/00; A61P 1/00

(52) U.S. Cl. CPC A61K 47/6803; A61K 31/282; A61P 35/00; C07K 16/32; A61P 15/00; A61P 1/00; C07K 2317/565; C07K 2317/90; A61K 2039/505

(57) ABSTRACT

Disclose herein are dosing regimens for a combination of carboplatin and a targeted NaPi2b antibody-drug conjugates for treating cancer.

### CROSS-REFERENCE TO RELATED APPLICATIONS

This application claims priority to, and the benefit of, U.S. Provisional Application No. 63/183,420 filed May 3, 2021 and U.S. Provisional Application No. 63/242,323 filed Sep. 9, 2021. The contents of each of these applications are hereby incorporated by reference in their entireties.

### INCORPORATION BY REFERENCE OF SEQUENCE LISTING

The contents of the text file named “MRSN-035_001US_SeqList.txt”, which was created on Apr. 29, 2022 and is 20 KB in size, are hereby incorporated by reference in their entirety.

### FIELD OF THE INVENTION

This disclosure relates generally to dosing regimens for administering a combination of NaPi2b targeted polymer antibody-drug conjugates and carboplatin for the treatment of high grade serous ovarian cancer.

### BACKGROUND OF THE INVENTION

NaPi2b (SLC34A2, NaPiIIb, Npt2), a multi-transmembrane, sodium-dependent phosphate transporter (Xu et al. Genomics 62:281-284 (1999)), is normally expressed at the brush border membrane of mammalian small intestine and participates in the transcellular inorganic phosphate (Pi) absorption, contributing to the maintenance of phosphate homeostasis in the body. The expression of NaPi2b at the protein level has been detected in the liver, at the apical surface of epithelial cells of mammary, salivary glands, and bronchi, and in the lungs, testis, thyroid gland, small intestine, and uterus. Mutations in NaPi2b have been associated with clinical syndromes of alveolar and testicular micro lithiasis. NaPi2b is highly expressed in non-squamous non-small cell lung cancer (NSCLC), non-mucinous ovarian cancer and papillary thyroid cancer. NaPi2b-positive tissue immunoreactivity is present in 61% of NSCLC and 92% of ovarian cancer specimens.

Ovarian cancer is one of the most common gynecologic malignancies and the fifth most frequent cause of cancer death in women. The high mortality rate results in part from the frequent diagnosis of ovarian cancer at advanced stages and the mortality rate is approximately 65% of the incidence rate. The constellation of diseases commonly referred to as “ovarian cancer,” includes epithelial ovarian, primary peritoneal and fallopian tube carcinomas and represents the most common cause of gynecologic cancer death in the United States. The lethality of this disease has been attributed largely to advanced stage at diagnosis (and absence of effective screening for potentially early-stage disease). In addition, after standard management of newly diagnosed advanced ovarian cancer (including surgical cytoreduction and platinum/taxane chemotherapy with or without the anti-VEGF monoclonal antibody bevacizumab, and with or without poly ADP-ribose polymerase (PARP) inhibitors (PARPi)), the vast majority of patients will experience recurrence and die of disease. The benefits of standard therapies are limited by both intrinsic and acquired drug resistance. Thus, there is a need for the development of new agents with activity against ovarian cancer, including those that target the biological activities of NaPi2b. Further, new combination therapies comprising an agent that targets NaPi2b in combination with one or more additional agents provide an important means of treating ovarian cancers.

### SUMMARY OF THE INVENTION

The disclosure provides a method of treating a platinum-sensitive recurrent high grade serous ovarian cancer in a subject in need thereof, comprising administering to the subject carboplatin at a target Area Under the Curve (AUC) of about 5 and a NaPi2b-targeted antibody polymer-drug conjugate by infusion at a dose of between 20 mg/m² to 43 mg/m² on the first day of treatment and every four weeks (i.e. 28 days) thereafter, wherein the NaPi2b-targeted antibody polymer-drug conjugate is:

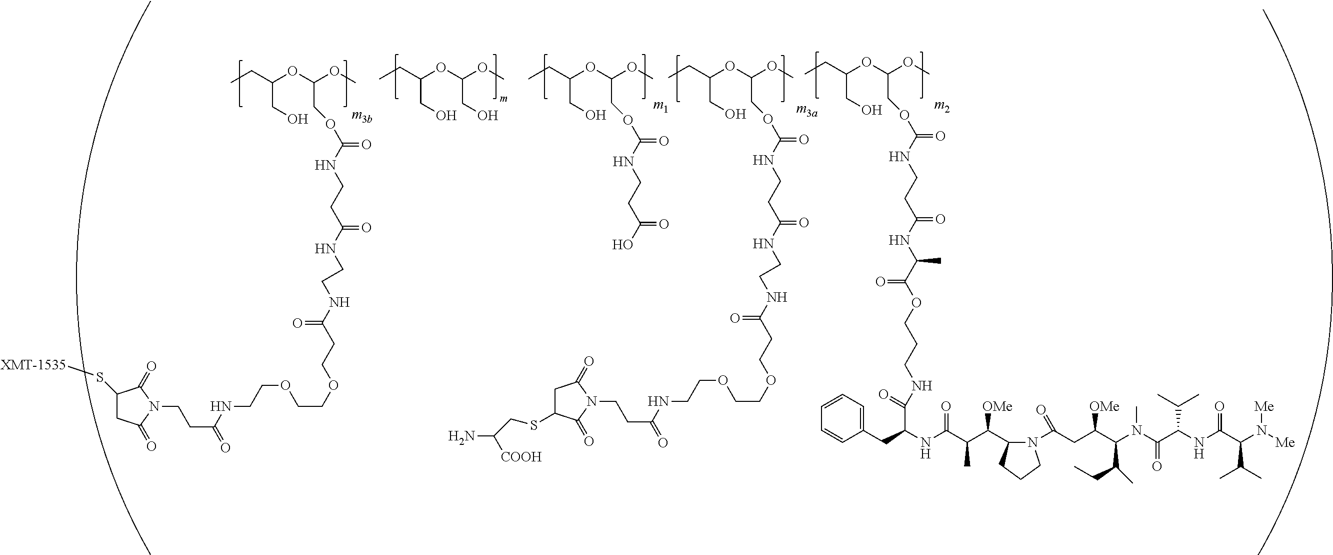

wherein the conjugate comprises a polymeric scaffold comprising poly(1-hydroxymethylethylene hydroxymethyl-formal) (PHF), wherein the PaF has a molecular weight ranging from 5 kDa to 10 kDa; m is an integer from 20 to 75, m₁ is an integer from about 5 to about 35, m₀ is an integer from about 3 to about 10, m₃ₐ is an integer from 0 to about 4, m_{3b} is an integer from 1 to about 5, the sum of m, m₁, m₂, m₃ₐ, and m_{3b} ranges from about 40 to about 75, the XMT-1535 antibody comprises a variable light chain complementarity determining region 1 (CDRL1) comprising the amino acid sequence SASQDIGNFLN (SEQ ID NO: 8); a variable light chain complementarity determining region 2 (CDRL2) comprising the amino acid sequence YTSSLYS (SEQ ID NO: 9); a variable light chain complementarity determining region 3 (CDRL3) comprising the amino acid sequence QQYSKLPLT (SEQ ID NO: 10); a variable heavy chain complementarity determining region 1 (CDRHI1) comprising the amino acid sequence GYTFTGYNIH (SEQ ID NO: 5); a variable heavy chain complementarity determining region 2 (CDRHI2) comprising the amino acid sequence AIYPGNGDTSYKQKFRG (SEQ ID NO: 6); and a variable heavy chain complementarity determining region 3 (CDRH3) comprising the amino acid sequence GETARATFAY (SEQ ID NO: 7); and wherein the ratio between the PHF and XMT-1535 is an integer from 2 to about 6.

In some embodiments, XMT-1535 comprises a variable heavy chain comprising the amino acid sequence of SEQ ID NO: 3 and a variable light chain comprising the amino acid sequence of SEQ ID NO: 4.

In some embodiments, XMT-1535 comprises a heavy chain comprising the amino acid sequence of SEQ ID NO: 1 and a light chain comprising the amino acid sequence of SEQ ID NO: 2.

In some embodiments, the conjugate dose is 20 mg/m². In some embodiments, the conjugate dose is 30 mg/m². In some embodiments, the conjugate dose is 36 mg/m². In some embodiments, the conjugate dose is 43 mg/m².

In some embodiments, the conjugate dose of 20 mg/m² is capped at BSA 2.2 m². In some embodiments, the conjugate dose of 30 mg/m² is capped at BSA 2.2 m². In some embodiments, the conjugate dose of 36 mg/m² is capped at BSA 2.2 m². In some embodiments, the conjugate dose of 43 mg/m² is capped at BSA 1.8 m². In some embodiments, the conjugate dose is 36 mg/m² up to a maximum of approximately 80 mg. In some embodiments, the conjugate dose is about 80 mg.

In some embodiments, the carboplatin is administered prior to the NaPi2b-targeted antibody polymer-drug conjugate.

In some embodiments, the high grade serous ovarian cancer is a metastatic high grade serous ovarian cancer. In some embodiments, the high grade serous ovarian cancer is a recurrent high grade serous ovarian cancer. In some embodiments, the high grade serous ovarian cancer is fallopian tube cancer or primary peritoneal cancer.

In some embodiments, the subject is administered carboplatin and the NaPi2b-targeted antibody polymer-drug conjugate on the first day of treatment and every 28 days (i.e. four weeks) thereafter for up to 6 cycles.

In some embodiments, after the subject is administered carboplatin and the NaPi2b-targeted antibody polymer-drug conjugate by infusion for up to 6 cycles, the subject is administered the NaPi2b-targeted antibody polymer-drug conjugate at a maintenance dose of 43 mg/m².

In some embodiments, after the subject is administered carboplatin and the NaPi2b-targeted antibody polymer-drug conjugate by infusion for up to 6 cycles, the subject is administered the NaPi2b-targeted antibody polymer-drug conjugate at a maintenance dose of 36 mg/m².

In some embodiments, after the subject is administered carboplatin and the NaPi2b-targeted antibody polymer-drug conjugate by infusion for up to 6 cycles, the subject is administered the NaPi2b-targeted antibody polymer-drug conjugate at a maintenance dose of 30 mg/m².

In some embodiments, after the subject is administered carboplatin and the NaPi2b targeted antibody polymer-drug conjugate by infusion for up to 6 cycles, the subject is administered the NaPi2b-targeted antibody polymer-drug conjugate at a maintenance dose of 20 mg/m².

In some embodiments, the maintenance dose of 20 mg/m² is capped at BSA 2.2 m². In some embodiments, the maintenance dose of 30 mg/m² is capped at BSA 2.2 m². In some embodiments, the maintenance dose of 36 mg/m² is capped at BSA 2.2 m². In some embodiments, the maintenance dose of 43 mg/m² is capped at BSA 1.8 m².

In some embodiments, PHF has a molecular weight ranging from about 5 kDa to about 10 kDa, m is an integer from 30 to about 35, m₁ is an integer from 8 to about 10, m₂ is an integer from 2 to about 5, m₃ₐ is an integer from 0 to about 1, m_{3b} is an integer from 1 to about 2, the sum of m₃ₐ and m_{3b} ranges from 1 and about 4, and the ratio between the PHF and the XMT-1535 antibody is about 3 to about 5.

In some embodiments, the ratio between m2 and XMT-1535 is about 16:1 to 10:1. In some embodiments, the ratio between m2 and XMT-1535 is about 12:1 to 8:1.

Other features and advantages of the invention will be apparent from the following detailed description and claims.

### DETAILED DESCRIPTION

The present disclosure provides methods of treating recurrent, platinum-sensitive high-grade serous ovarian cancer (HGSOC), by administration of a combination of carboplatin and a NaPi2b-targeted polymer antibody-drug conjugate (XMT-1536) that specifically binds to the extracellular region of SLC34A2. Specifically, the invention provides dosing regimens for the combination of carboplatin XMT-1536 in the treatment of NaPi2b expressing ovarian cancers by administration as an intravenous infusion. XMT-1536 is comprised of about 8-12 molecules of auristatin F-hydroxypropyl amide (AF HPA) conjugated to a cysteine moiety of a NaPi2b monoclonal antibody (XMT-1535) via a poly(1-hydroxymethylethylene hydroxymethyl-formal) (PHF) scaffold.

Patients with recurrent, platinum-sensitive HGSOC, including fallopian tube and primary peritoneal cancer, are intravenously administered a combination of carboplatin and XMT-1536 once every 4 weeks (i.e. 28 days). Accordingly, the invention features methods of treating platinum-sensitive HGSOC by administering to a subject, i.e., human, in a dose escalation study an infusion dose of carboplatin at a target AUC of 5 that avoids unacceptable toxicities, followed by administration of XMT-1536 at 20 mg/m², 30 mg/m², 36 mg/m² or 43 mg/m² once every 4 weeks (i.e. 28 days) for 6 cycles with a cap of body surface area (BSA) at 1.8 m² for the 43 mg/m² dose and a cap of BSA at 2.2 m² for the 20 mg/m², 30 mg/m² and 36 mg/m² doses. After completion of the last dose of the combination therapy, the subject is administered a maintenance dose of XMT-1536 monotherapy until disease progression, unacceptable toxicity, or voluntary discontinuation.

In some embodiments the subject has been identified as having NaPi2b expression. In some embodiments, the NaPi2b expression is in the form of a NaPi2b expressing tumor. NaPi2b expression is detected by methods known in the art. For example, by immunohistochemistry (IHC) analysis, fluorescent in situ hybridization (FISH) assay or RNA expression analysis of NaPi2b transcript or other genes related to cancer measured in tumor samples. Blood-based biomarkers, which may include serum cytokines, circulating immune cells, and circulating tumor cells can also be used to determine the NaPi2b expression levels.

### NaPi2b Antibodies

The NaPi2b antibodies suitable for the methods of the disclosure specifically bind to the extracellular region of SLC34A2. The disclosure further provides NaPi2b-targeted monoclonal antibodies that specifically recognize NaPi2b, also known as sodium-dependent phosphate transport protein 2B. The NaPi2b antibodies used in the conjugates disclosed herein are capable of and useful in modulating, e.g., blocking, inhibiting, reducing, antagonizing, neutralizing or otherwise interfering with at least one biological activity of NaPi2b. Antibodies disclosed herein also include antibodies that bind soluble NaPi2b. The NaPi2b antibodies specifically bind to an epitope on an extracellular domain (ECD) of the human NaPi2b. These antibodies are collectively referred to herein as “NaPi2b” antibodies.

The NaPi2b antibody-drug conjugates provided herein include antibodies that bind to a NaPi2b epitope with an equilibrium dissociation constant (K_{d} or K_{D}) of ≤1 μM, e.g., ≤100 nM, preferably ≤10 nM, and more preferably ≤1 nM. For example, the NaPi2b antibodies used in the antibody-drug conjugates disclosed herein exhibit a K_{d} in the range approximately between ≤1 nM to about 1 pM.

The NaPi2b antibody-drug conjugates provided herein can include antibodies that serve to modulate, block, inhibit, reduce, antagonize, neutralize or otherwise interfere with the functional activity of NaPi2b. Functional activities of NaPi2b include for example, participating in the transcellular inorganic phosphate (Pi) absorption, thereby contributing to the maintenance of phosphate homeostasis in the body. For example, the NaPi2b antibodies completely or partially inhibit NaPi2b functional activity by partially or completely modulating, blocking, inhibiting, reducing antagonizing, neutralizing, or otherwise interfering with transcellular inorganic phosphate absorption. Transcellular inorganic phosphate absorption activity is assessed using any art-recognized method for detecting transcellular inorganic phosphate absorption activity, including, but not limited to detecting levels of transcellular inorganic phosphate absorption in the presence and absence of an anti-NaPi2b antibody disclosed herein.

The NaPi2b antibodies are considered to completely modulate, block, inhibit, reduce, antagonize, neutralize or otherwise interfere with NaPi2b functional activity when the level of NaPi2b functional activity in the presence of the NaPi2b antibody is decreased by at least 95%, e.g., by 96%, 97%, 98%, 99% or 100% as compared to the level of NaPi2b functional activity in the absence of binding with a NaPi2b antibody described herein. The NaPi2b antibodies are considered to partially modulate, block, inhibit, reduce, antagonize, neutralize or otherwise interfere with NaPi2b functional activity when the level of NaPi2b activity in the presence of the NaPi2b antibody is decreased by less than 95%, e.g., 10%, 20%, 25%, 30%, 40%, 50%, 60%, 75%, 80%, 85% or 90%, as compared to the level of NaPi2b activity in the absence of binding with a NaPi2b antibody described herein.

Exemplary antibodies disclosed herein include, the XMT-1535 antibody. These antibodies show specificity for human NaPi2b and they have been shown to inhibit NaPi2b activity.

NaPi2b human or humanized monoclonal antibody, XMT-1535, includes a heavy chain (HC), heavy chain variable region (VH), light chain (LC), and a light chain variable region (VL), as shown in the amino acid and corresponding nucleic acid sequences presented below. The variable heavy chain region and variable light chain region for each antibody are shaded in the amino acid sequences below. The complementarity determining regions (CDRs) of the heavy chain and the light chain are underlined in the amino acid sequences presented below. The amino acids encompassing the complementarity determining regions (CDRs) for the XMT-1535 antibody are as defined by E. A. Kabat et al. (See Kabat, E. A., et al., Sequences of Protein of immunological interest, Fifth Edition, US Department of Health and Human Services, US Government Printing Office (1991)) and are disclosed in U.S. Pat. No. 8,603,474.

>XMT-1535 Heavy Chain Amino Acid Sequence (Heavy chain variable region (SEQ ID NO: 3)
(Italicized) + IgG1 Heavy chain constant region (SEQ ID NO: 11))
(SEQ ID NO: 1)

[symbol]

[symbol]

[symbol]

[symbol]

[symbol]

[symbol]

A
STKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSPG*
CDRH1:
(SEQ ID NO: 5)
GYTFTGYNIH
CDRH2:
(SEQ ID NO: 6)
AIYPGNGDTSYKQKFRG
CDRH3:
(SEQ ID NO: 7)
GETARATFAY
>XMT-1535 Heavy chain variable region nucleic acid sequence
CAAGTTCAGCTGGTTCAGTCTGGCGCCGAGGTTGTGAAACCTGGCGCCTCTGTGAAGAT
GAGCTGCAAGGCCAGCGGCTACACCTTCACCGGCTACAACATCCACTGGGTCAAGCAG
GCCCCTGGACAGGGACTCGAATGGATCGGAGCCATCTATCCCGGCAACGGCGACACCA
GCTACAAGCAGAAGTTCCGGGGCAGAGCCACACTGACCGCCGATACAAGCACCAGCA
CCGTGTACATGGAACTGAGCAGCCTGAGAAGCGAGGACAGCGCCGTGTACTATTGCGC
CAGAGGCGAAACAGCCAGAGCCACCTTTGCCTATTGGGGCCAGGGAACCCTGGTCACC
GTTAGCTCT
>XMT-1535 Light Chain Amino Acid Sequence (Light chain variable region (SEQ ID NO: 4)
(Italicized) + Light chain constant region (SEQ ID NO: 12))
(SEQ ID NO: 2)

[symbol]

[symbol]

[symbol]

[symbol]

[symbol]

RTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
CDRL1:
(SEQ ID NO: 8)
SASQDIGNFLN
CDRL2:
(SEQ ID NO: 9)
YTSSLYS
CDRL3:
(SEQ ID NO: 10)
QQYSKLPLT
>XMT-1535 Light chain variable region nucleic acid sequence
(SEQ ID NO: 14)
GATATTCAGATGACACAGAGCCCCAGCAGCCTGTCTGCCTCTGTGGGAGACAGAGTGA
CCATCACCTGTAGCGCCAGCCAGGATATCGGCAACTTCCTGAACTGGTATCAGCAGAA
ACCCGGCAAGACCGTGAAGGTGCTGATCTACTACACCTCCAGCCTGTACAGCGGCGTG
CCCAGCAGATTTTCTGGCAGCGGCTCTGGCACCGACTACACCCTGACCATATCTAGCCT
GCAGCCTGAGGACTTCGCCACCTACTACTGCCAGCAGTACAGCAAGCTGCCCCTGACA
TTTGGCCAGGGCACCAAGCTGGAACTGAAG

Also included in the disclosure are antibodies that bind to the same epitope or cross compete for binding to the same epitope as the antibodies described herein. For example, antibodies disclosed herein specifically bind to NaPi2b, wherein the antibody binds to an epitope that includes one or more amino acid residues on human NaPi2b (e.g., GenBank Accession No. 095436.3).

Antibodies disclosed herein specifically bind to an epitope on the full-length human NaPi2b comprising the amino acid sequence:

| (SEQ ID NO: 15) | |
|---|---|
| 1 | MAPWPELGDA QPNPDKYLEG AAGQQPTAPD KSKETNKTDN TEAPVTKIEL |
| 51 | LPSYSTATLI DEPTEVDDPW NLPTLQDSGI KWSERDTKGK ILCFFQGIGR |
| 101 | LILLLGFLYF FVCSLDILSS AFQLVGGKMA GQFFSNSSIM SNPLLGLVIG |
| 151 | VLVTVLVQSS STSTSIVVSM VSSSLLTVRA AIPIIMGANI GTSITNTIVA |
| 201 | LMQVGDRSEF RRAFAGATVH DFFNWLSVLV LLPVEVATHY LEIITQLIVE |
| 251 | SFHFKNGEDA PDLLKVITKP FTKLIVQLDK KVISQIAMND EKAKNKSLVK |
| 301 | IWCKTFTNKT QINVTVPSTA NCTSPSLCWT DGIQNWTMKN VTYKENIAKC |
| 351 | QHIFVNFHLP DLAVGTILLI LSLLVLCGCL IMIVKILGSV LKGQVATVIK |
| 401 | KTINTDFPFP FAWLTGYLAI LVGAGMTFIV QSSSVFTSAL TPLIGIGVIT |
| 451 | IERAYPLTLG SNIGTTTTAI LAALASPGNA LRSSLQIALC HFFFNISGIL |
| 501 | LWYPIPFTRL PIRMAKGLGN ISAKYRWFAV FYLIIFFFLI PLTVFGLSLA |
| 551 | GWRVLVGVGV PVVFIIILVL CLRLLQSRCP RVLPKKLQNW NFLPLWMRSL |
| 601 | KPWDAVVSKF TGCFQMRCCC CCRVCCRACC LLCDCPKCCR CSKCCEDLEE |
| 651 | AQEGQDVPVK APETFDNITI SREAQGEVPA SDSKTECTAL |

Antibodies disclosed herein specifically bind to an epitope on an extracellular domain (ECD) of the human NaPi2b.

Those skilled in the art will recognize that it is possible to determine, without undue experimentation, if a monoclonal antibody has the same specificity as a monoclonal antibody disclosed herein (e.g., XMT-1535) by ascertaining whether the former prevents the latter from binding to a natural binding partner or other molecule known to be associated with NaPi2b. If the monoclonal antibody being tested competes with the monoclonal antibody disclosed herein, as shown by a decrease in binding by the monoclonal antibody disclosed herein, then the two monoclonal antibodies bind to the same, or a closely related, epitope.

An alternative method for determining whether a monoclonal antibody has the specificity of monoclonal antibody disclosed herein is to pre-incubate the monoclonal antibody disclosed herein with soluble NaPi2b (with which it is normally reactive), and then add the monoclonal antibody being tested to determine if the monoclonal antibody being tested is inhibited in its ability to bind NaPi2b. If the monoclonal antibody being tested is inhibited then, in all likelihood, it has the same, or functionally equivalent, epitopic specificity as the monoclonal antibody disclosed herein.

Screening of monoclonal antibodies disclosed herein, can also be carried out, e.g., by measuring NaPi2b-mediated activity, and determining whether the test monoclonal antibody is able to modulate, block, inhibit, reduce, antagonize, neutralize or otherwise interfere with NaPi2b activity.

The antibodies disclosed herein contain a heavy chain variable region having an amino acid sequence at least 90%, 91%, 92%, 93%, 94%, 95%, 96%, 97%, 98%, 99% or more identical to a sequence selected from the group consisting of SEQ ID NOs: 3 and a light chain variable region having an amino acid sequence at least 90%, 91%, 92%, 93%, 94%, 95%, 96%, 97%, 98%, 99% or more identical to a sequence selected from the group consisting of SEQ ID NOs: 4.

In some embodiments, the antibodies disclosed herein contain a heavy chain amino acid sequence at least 90%, 91%, 92%, 93%, 94%, 95%, 96%, 97%, 98%, 99% or more identical to the amino acid sequence of SEQ ID NO: 1 and a light chain amino acid sequence at least 90%, 91%, 92%, 93%, 94%, 95%, 96%, 97%, 98%, 99% or more identical to the amino acid sequence of SEQ ID NO: 2.

The antibodies disclosed herein contain a heavy chain variable region having an amino acid sequence at least 85%, 86%, 87%, 88%, 89%, 90%, 91%, 92%, 93%, 94%, 95%, 96%, 97%, 98%, 99% or more identical to a sequence selected from the group consisting of SEQ ID NOs: 3 and a light chain variable region having an amino acid sequence at least 85%, 86%, 87%, 88%, 89%, 90%, 91%, 92%, 93%, 94%, 95%, 96%, 97%, 98%, 99% or more identical to a sequence selected from the group consisting of SEQ ID NOs: 4.

In some embodiments, the antibodies disclosed herein contain a heavy chain amino acid sequence at least 85%, 86%, 87%, 88%, 89%, 90%, 91%, 92%, 93%, 94%, 95%, 96%, 97%, 98%, 99% or more identical to the amino acid sequence of SEQ ID NO: 1 and a light chain amino acid sequence at least 85%, 86%, 87%, 88%, 89%, 90%, 91%, 92%, 93%, 94%, 95%, 96%, 97%, 98%, 99% or more identical to the amino acid sequence of SEQ ID NO: 2.

In some embodiments, the antibodies disclosed herein contain the heavy chain variable region amino acid sequence of SEQ ID NO: 3 and the light chain variable region amino acid sequence of SEQ ID NO: 4.

In some embodiments, the antibodies disclosed herein contain the heavy chain amino acid sequence of SEQ ID NO: 1 and the light chain amino acid sequence of SEQ ID NO: 2.

In some embodiments, the antibodies disclosed herein contain the CDRH1 amino acid sequence of SEQ ID NO: 5, the CDRH2 amino acid sequence of SEQ ID NO: 6, the CDRH3 amino acid sequence of SEQ ID NO: 7, the CDRL1 amino acid sequence of SEQ ID NO: 8, the CDRL2 amino acid sequence of SEQ ID NO: 9, and the CDRL3 amino acid sequence of SEQ ID NO: 10.

In some embodiments, the antibodies disclosed herein that contains the amino acid sequence at least 90%, 91%, 92%, 93%, 94%, 95%, 96%, 97%, 98%, 99% or more identical to the amino acid sequence GYTFTGYNIH (SEQ ID NO: 5); a CDRH2 that contains the amino acid sequence at least 90%, 91%, 92%, 93%, 94%, 95%, 96%, 97%, 98%, 99% or more identical to the amino acid sequence AIYPGNGDTSYKQKFRG (SEQ ID NO: 6); a CDRH3 that contains the amino acid sequence at least 90%, 91%, 92%, 93%, 94%, 95%, 96%, 97%, 98%, 99% or more identical to the amino acid sequence GETARATFAY (SEQ ID NO: 7); a CDRL1 that contains the amino acid sequence at least 90%, 91%, 92%, 93%, 94%, 95%, 96%, 97%, 98%, 99% or more identical to the amino acid sequence SASQDIGNFLN (SEQ ID NO: 8); a CDRL2 that contains the amino acid sequence at least 90%, 91%, 92%, 93%, 94%, 95%, 96%, 97%, 98%, 99% or more identical to the amino acid sequence YTSSLYS (SEQ ID NO: 9); and a CDRL3 that contains the amino acid sequence at least 90%, 91%, 92%, 93%, 94%, 95%, 96%, 97%, 98%, 99% or more identical to the amino acid sequence QQYSKLPLT (SEQ ID NO: 10).

In some embodiments, the antibodies disclosed herein that contains the amino acid sequence at least 85%, 86%, 87%, 88%, 89%, 90%, 91%, 92%, 93%, 94%, 95%, 96%, 97%, 98%, 99% or more identical to the amino acid sequence GYTFTGYNIH (SEQ ID NO: 5); a CDRH2 that contains the amino acid sequence at least 85%, 86%, 87%, 88%, 89%, 90%, 91%, 92%, 93%, 94%, 95%, 96%, 97%, 98%, 99% or more identical to the amino acid sequence AIYPGNGDTSYKQKFRG (SEQ ID NO: 6); a CDRH3 that contains the amino acid sequence at least 85%, 86%, 87%, 88%, 89%, 90%, 91%, 92%, 93%, 94%, 95%, 96%, 97%, 98%, 99% or more identical to the amino acid sequence GETARATFAY (SEQ ID NO: 7); a CDRL1 that contains the amino acid sequence at 85%, 86%, 87%, 88%, 89%, 90%, 91%, 92%, 93%, 94%, 95%, 96%, 97%, 98%, 99% or more identical to the amino acid sequence SASQDIGNFLN (SEQ ID NO: 8); a CDRL2 that contains the amino acid sequence at least 85%, 86%, 87%, 88%, 89%, 90%, 91%, 92%, 93%, 94%, 95%, 96%, 97%, 98%, 99% or more identical to the amino acid sequence YTSSLYS (SEQ ID NO: 9); and a CDRL3 that contains the amino acid sequence at least 85%, 86%, 87%, 88%, 89%, 90%, 91%, 92%, 93%, 94%, 95%, 96%, 97%, 98%, 99% or more identical to the amino acid sequence QQYSKLPLT (SEQ ID NO: 10).

In certain embodiments, the antibodies disclosed herein include one or more conservative amino acid substitutions in a variable domain sequence such as 1, 2, 3, 4, 5, 6, 7, 8, 9, 10, 11, 12, 13, 14, 15, or more conservative substitutions in a variable domain sequence. In some embodiments, these conservative amino acid substitutions are in a CDR region, e.g., 1, 2, 3, 4, 5, 6, 7, 8, 9, 10, 11, 12, 13, 14, 15, or more conservative substitutions are made cumulatively across all CDRs and in some particular embodiments, up to 1, 2, 3, or 4 conservative amino acid substitutions may be present in each CDR sequence, e.g., SEQ ID NOs: 5-10.

Those skilled in the art will recognize that it is possible to determine, without undue experimentation, if a monoclonal antibody has the same specificity as a monoclonal antibody XMT-1535, by ascertaining whether the former prevents the latter from binding to a natural binding partner or other molecule known to be associated with NaPi2b. If the monoclonal antibody being tested competes with the monoclonal antibody disclosed herein, as shown by a decrease in binding by the monoclonal antibody disclosed herein, then the two monoclonal antibodies bind to the same, or a closely related, epitope.

An alternative method for determining whether a monoclonal antibody has the specificity of monoclonal antibody disclosed herein is to pre-incubate the monoclonal antibody disclosed herein with soluble NaPi2b (with which it is normally reactive), and then add the monoclonal antibody being tested to determine if the monoclonal antibody being tested is inhibited in its ability to bind NaPi2b. If the monoclonal antibody being tested is inhibited then, in all likelihood, it has the same, or functionally equivalent, epitopic specificity as the monoclonal antibody disclosed herein.

Screening of monoclonal antibodies disclosed herein, can be also carried out, e.g., by measuring NaPi2b-mediated activity, and determining whether the test monoclonal antibody is able to modulate, block, inhibit, reduce, antagonize, neutralize or otherwise interfere with NaPi2b activity.

The NaPi2b antibodies suitable for use in the methods disclosed herein can be generated and purified by well-known techniques e.g., WO 2009/097128, WO 2017/160754, and U.S. Ser. No. 16/136,706, each of which is incorporated herein in its entirety by reference.

### NaPi2b Targeted Polymer Antibody Drug Conjugates

The invention pertains to therapies involving immunoconjugates comprising an antibody conjugated to a cytotoxic agent such as a toxin (e.g., an enzymatically active toxin of bacterial, fungal, plant, or animal origin, or fragments thereof), via a polymer scaffold.

The conjugate described herein includes a NaPi2b antibody connected to one or more AF-HPA-carrying polymeric scaffolds independently comprising poly(1-hydroxymethylethylene hydroxymethyl-formal) (PHF) having a molecular weight ranging from about 5 kDa to about 10 kDa. The AF-HPA-carrying polymeric scaffold is conjugated to the NaPi2b targeted antibody via the NaPi2b cysteine residues.

Specifically, the NaPi2b targeted polymer antibody-drug conjugate is XMT-1536 and has the Formula (A):

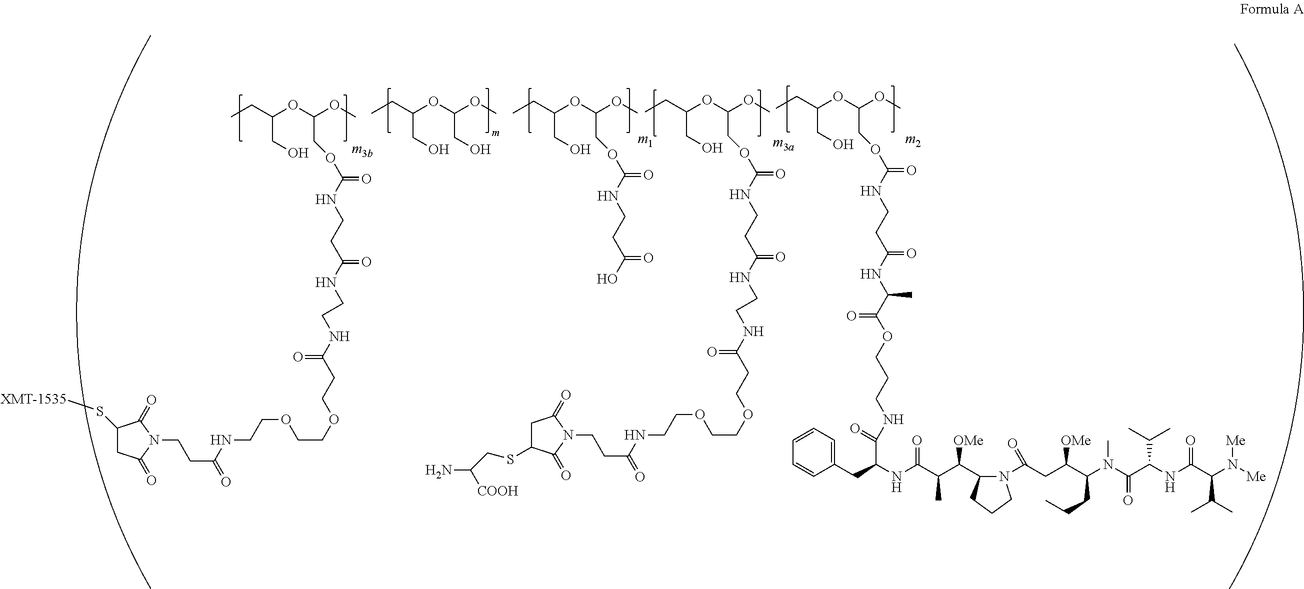

wherein.

the polymer comprises poly(1-hydroxymethylethylene hydroxymethyl-formal) (PHF) having a molecular weight ranging from about 5 kDa to about 10 kDa;

m is an integer from 20 to 75,

m₁ is an integer from about 5 to about 35,

m₂ is an integer from about 3 to about 10,

m₃ₐ is an integer from 0 to about 4,

m_{3b} is an integer from 1 to about 5,

the sum of m, m₁, m₂, m₃ₐ, and m_{3b} ranges from about 40 to about 75,

the ratio between the PHF and the XMT-1535 antibody is about 2 to about 6, and XMT-1535 is the fully human or humanized NaPi2b antibody XMT-1535 described herein.

In some embodiments, m is an integer from about 30 to about 75.

In some embodiments, m is an integer from about 30 to about 40.

In some embodiments, m₁ is an integer from about 10 to about 20.

In some embodiments, m₁ is an integer from about 10 to about 12.

In some embodiments, m₂ is an integer from about 3 to about 5.

In some embodiments, m₃ₐ is an integer from 0 to about 1.

In some embodiments, m_{3b} is an integer from 2 to about 4

In some embodiments, the ratio between the PHF and the XMT-1535 antibody is about 2 to about 5.

In some embodiments, the ratio between the PHF and the XMT-1535 antibody is about 2 to about 4.

In some embodiments, the ratio between the PHF and the XMT-1535 antibody is about 3 to about 5.

In some embodiments, the ratio between the PHF and the XMT-1535 antibody is about 3 to about 4.

In some embodiments the NaPi2b targeted polymer antibody-drug conjugate comprises 10-15 molecules of AF-HPA.

In some embodiments, the PHF has a molecular weight ranging from about 6 kDa to about 8 kDa.

In some embodiments, the PHF has a molecular weight ranging from about 6 kDa to about 7 kDa.

In certain embodiments, the NaPi2b targeted polymer antibody-drug conjugate Formula (A) is of Formula (B), wherein the polymer is PHF that has a molecular weight ranging from about 5 kDa to about 10 kDa:

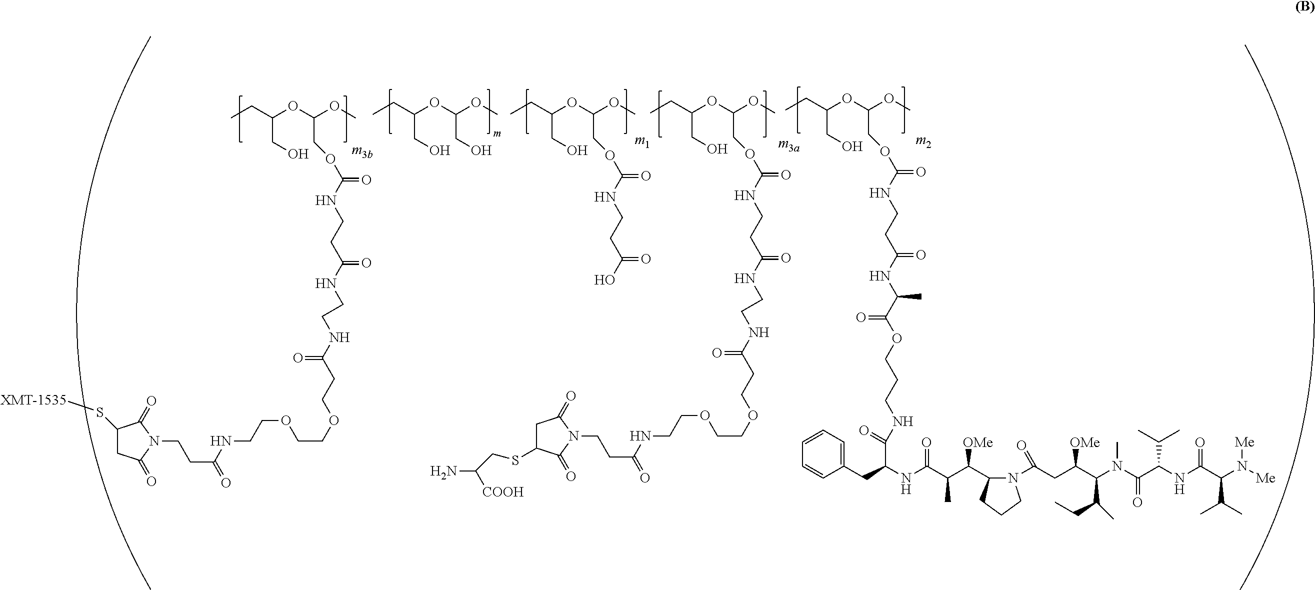

wherein:

m is an integer from 30 to about 35,

m₁ is an integer from 8 to about 10,

m₂ is an integer from 2 to about 5,

m₃ₐ is an integer from 0 to about 1,

m_{3b} is an integer from 1 to about 2,

the sum of m₃ₐ and m_{3b} ranges from 1 and about 4, and

the ratio between the PHF and the XMT-1535 antibody is about 3 to about 5.

The NaPi2b targeted polymer antibody-drug conjugates, (i.e., XMT-1536) suitable for use in the methods disclosed herein can be generated and purified by well-known techniques e.g., WO 2009/097128, WO 2017/160754, PCT/US18/38988 and U.S. Ser. No. 16/136,706, each of which is incorporated herein in its entirety by reference.

### Dosage and Administration

The high grade serous ovarian cancer therapy provided herein, comprises a combination of carboplatin and NaPi2b-targeted polymer antibody-drug conjugate (XMT-1536), administered in an amount sufficient to exert a therapeutically useful effect. Typically, the active agents are administered in an amount that does not result in undesirable side effects of the patient being treated, or that minimizes or reduces the observed side effects. The high grade serous ovarian cancer includes, but is not limited to, fallopian tube cancer and primary peritoneal cancer.

It is within the level of one of skill in the art to determine the precise amounts of active agents, including carboplatin and XMT-1536 to be administered to a subject. For example, such agents and uses for treating cancers and solid tumors, are well-known in the art. Thus, dosages of such agents can be chosen based on standard dosing regimens for that agent under a given route of administration.

In some embodiments, the subject has platinum-sensitive HGSOC. In some embodiments the subject has fallopian tube cancer. In other embodiments, the subject has primary peritoneal cancer. The platinum-sensitive HGSOC can be recurrent. In some embodiments the subject has a recurrence of high grade serous ovarian cancer, that includes fallopian tube cancer and primary peritoneal cancer.

In some embodiments, the subject having platinum-sensitive HGSOC is administered carboplatin and XMT-1536. In some embodiments, carboplatin is administered first followed by administration of XMT-1536. In some embodiments, XMT-1536 is administered first followed by administration of carboplatin. In some embodiments, carboplatin and XMT-1536 are administered simultaneously.

In some embodiments, administration of carboplatin and XMT-1536 is via infusion. Methods of infusion can comprise any method of infusing therapeutic agents to a subject known in the art. In some embodiments the infusion is an intravenous (IV) infusion.

In some embodiments, infusions of carboplatin occur over a duration of at least 1 minute, at least 5 minutes, at least 10 minutes, at least 15 minutes, at least 20 minutes, at least 25 minutes, at least 30 minutes, at least 35 minutes, at least 45 minutes, at least 50 minutes, at least 55 minutes, at least 60 minutes, at least 65 minutes, at least 70 minutes, at least 80 minutes, at least 90 minutes, at least 100 minutes, at least 110 minutes, at least 120 minutes, or any number of minutes therebetween. In some embodiments, the duration of infusion can be varied from the first infusion to the second and subsequent infusions.

In some embodiments, infusions of XMT-1536 occur over a duration of at least 1 minute, at least 5 minutes, at least 10 minutes, at least 15 minutes, at least 20 minutes, at least 25 minutes, at least 30 minutes, at least 35 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, or any number of minutes therebetween. In some embodiments, the duration of infusion can be varied from the first infusion to the second and subsequent infusions.

The subject having platinum-sensitive HGSOC is administered carboplatin over 30 minutes by infusion, after which, following 30±5 minutes of monitoring for infusion-related reaction (IRR), XMT-1536 is then administered. XMT-1536 will be administered over 90 min for the first infusion, then over 30±5 minutes for the subsequent infusions for up to 6 cycles once every 4 weeks (i.e. 28 days).

In some embodiments the subject is administered carboplatin over 30 minutes at a target AUC of 5 by infusion thereby avoiding unacceptable dose-limiting toxicities. The subject is then administered by infusion XMT-1536 at a dosage amount that is between about 20 mg/m² to 43 mg/m². For example, the dosage of XMT-1536 is 20 mg/m². Alternatively, the dosage of XMT-1536 is 30 mg/m². In some embodiments, the dosage of XMT-1536 is 36 mg/m². In other embodiments, the dosage of XMT-1536 is 43 mg/m². In some embodiments, the dosages of XMT-1536 of 20 mg/m², 30 mg/m² and 36 mg/m² each have a cap of BSA at 2.2 m². In some embodiments, the dosages of XMT-1536 of 43 mg/m² has a cap of BSA at 1.8 m². In some embodiments, the dosage of XMT-1536 is 36 mg/m² up to a maximum of approximately 80 mg. In some embodiments, the dosage of XMT-1536 is about 80 mg. In these embodiments the dosage amounts are administered intravenously once every four weeks i.e. 28-day cycle after the administration of carboplatin at a target AUC of 5 by infusion. In these embodiments the carboplatin and XMT-1536 is administered for up to 6 cycles once every four weeks i.e. 28-days.

In some embodiments the subject is administered carboplatin over 30 minutes at a target AUC of 5 by infusion following which the subject is administered XMT-1536 at a dosage of 20 mg/m² over 90 min for the first infusion, then over 30 minutes for the subsequent infusions for up to 6 cycles once every 4 weeks i.e. 28-days.

In some embodiments the subject is administered carboplatin over 30 minutes at a target AUC of 5 by infusion following which the subject is administered XMT-1536 at a dosage of 30 mg/m² over 90 min for the first infusion, then over 30 minutes for the subsequent infusions for up to 6 cycles once every 4 weeks i.e. 28-days.

In some embodiments the subject is administered carboplatin over 30 minutes at a target AUC of 5 by infusion following which the subject is administered XMT-1536 at a dosage of 36 mg/m² over 90 min for the first infusion, then over 30 minutes for the subsequent infusions for up to 6 cycles once every 4 weeks i.e. 28-days.

In some embodiments the subject is administered carboplatin over 30 minutes at a target AUC of 5 by infusion following which the subject is administered XMT-1536 at a dosage of 43 mg/m². over 90 min for the first infusion, then over 30 minutes for the subsequent infusions for up to 6 cycles once every 4 weeks i.e. 28-days.

In some embodiments the subject is administered carboplatin over 30 minutes at a target AUC of 5 by infusion following which the subject is administered XMT-1536 at a dosage of 36 mg/m² over 30 minutes for the infusions for up to 5 cycles once every 4 weeks i.e. 28-days.

In some embodiments the subject is administered carboplatin over 30 minutes at a target AUC of 5 by infusion following which the subject is administered XMT-1536 at a dosage of 30 mg/m² over 30 minutes for the infusions for up to 5 cycles once every 4 weeks i.e. 28-days.

In some embodiments the subject is administered carboplatin over 30 minutes at a target AUC of 5 by infusion following which the subject is administered XMT-1536 at a dosage of 20 mg/m² over 30 minutes for the infusions for up to 5 cycles once every 4 weeks i.e. 28-days.

In some embodiments, after the administration of the combination of carboplatin and XMT-1536 by infusions for up to 6 cycles once every 4 weeks i.e. 28-days, the subject is administered XMT-1536 as a maintenance monotherapy until disease progression, unacceptable toxicity, voluntary withdrawal or death occurs.

In some embodiments, the maintenance monotherapy comprises XMT-1536 infusions at a dosage amount that is between about 20 mg/m² to 43 mg/m². In some embodiments, the maintenance dose is 43 mg/m². In some embodiments, the maintenance dose is 36 mg/m². In some embodiments, the maintenance dose is 30 mg/m². In some embodiments, the maintenance dose is 20 mg/m². In some embodiments, the maintenance dosages of XMT-1536 of 20 mg/m², 30 mg/m² and 36 mg/m² each have a cap of BSA at 2.2 m². In some embodiments, the maintenance dose of XMT-1536 of 43 mg/m² dose has a cap of BSA at 1.8 m². In some embodiments the subject is administered XMT-1536 at a dose of about 80 mg over 90 min for the first infusion, then over 30 minutes for the subsequent infusions for up to 18 cycles once every 4 weeks i.e. 28-days.

In some embodiments, the maintenance monotherapy is administered as an infusion every one week i.e. 7-days, every two weeks i.e. 14-days, every three weeks i.e. 21-days, every four weeks i.e. 28-days, every five weeks i.e. 35-days, every six weeks i.e. 42-days, every seven weeks i.e. 49-day, or every eight weeks i.e. 56-days.

### Measurement of NaPi2b Expression

In various embodiments the invention provides a method for identifying a cancer patient amenable to NaPi2b targeted therapy or monitoring the treatment regimen by measuring the status of NaPi2b expression in a tumor sample obtained from the patient.

In some embodiments, the NaPi2b diagnostic tests can be used to identify subjects for treatment with the NaPi2b targeted polymer drug conjugate.

The sample is derived from the subject having a cancer. The sample of cancer cells is dissected from tissue removed or obtained from the subject. In some embodiments, the sample is a fresh, frozen or an archival biopsy sample.

In some embodiments, the test cell population is derived from fresh, unfrozen tissue from a biopsy sample. In other embodiments, the test cell population is derived from a primary or metastatic site. In some embodiments, the test cell population is derived from a fresh or frozen tissue from a biopsy or surgical sample or ascitic fluid or pleural fluid. In some embodiments, the test cell population is derived from a fixed tissue (e.g., formalin fixation or formalin-fixed paraffin-embedded (FFPE)) from a biopsy or surgical sample or cell block derived from a fluid specimen. The tissue sample may be frozen or fresh.

The requisite level of NaPi2b expression may be that which is identified by the any methods known in the art and more specifically by the methods described herein. For example, the level of NaPi2b expression can be measured by conducting a known immunological assay, such as an enzyme immunoassay, radioimmunoassay, competitive immunoassay, double antibody sandwich assay, fluoroimmuno assay, ELISA, Western blotting technique, agglutination assay, cytofluorometry (e.g. flow cytometry), Fluorescence in situ hybridization (FISH), colorimetric or immunohistochemical staining assay (IHC) for protein expression using an antibody that specifically recognizes NaPi2b. Cell-based assays, such as, for example, flow cytometry (FC), immuno-histochemistry (IHC), RNA expression analysis or immunofluorescence (IF) are particularly desirable in determining NaPi2b expression status, since such assay formats are clinically-suitable.

Flow cytometry (FC) may be employed to determine cell surface expression of NaPi2b in a tumor sample before, during, and after treatment with a drug. For example, tumor cells may be analyzed by flow cytometry for NaPi2b expression, as well as for markers identifying cancer cell types, etc., if so desired. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 2% paraformaldehyde for 10 minutes at 37° C. followed by permeabilization in 90% methanol for 30 minutes on ice. Cells may then be stained with NaPi2b-specific antibody, washed and labeled with a fluorescent-labeled secondary antibody. The cells would then be analyzed on a flow cytometer (e.g. a Beckman Coulter FC500) according to the specific protocols of the instrument used. Such an analysis would identify the level of expressed NaPi2b in the tumor.

Immunohistochemical (IHC) staining may be also employed to determine the expression of NaPi2b in a tumor sample before, during, and after treatment with a drug. IHC may be carried out according to well-known techniques. See, e.g., ANTIBODIES; A LABORATORY MANUAL, Chapter 10, Harlow & Lane Eds., Cold Spring Harbor Laboratory (1988). Briefly, and by way of example, paraffin-embedded tissue (e.g. tumor tissue from a biopsy) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary polypeptide antibody and secondary antibody; and finally detecting using ABC avidin/biotin method according to manufacturer's instructions.

Immunofluorescence (IF) assays may be also employed to determine the expression of NaPi2b tumor sample before, during, and after treatment with a drug. IF may be carried out according to well-known techniques. See, e.g., J. M. Polak and S. Van Noorden (1997) INTRODUCTION TO IMMUNOCYTOCHEMISTRY, 2nd Ed.; ROYAL MICROSCOPY SOCIETY MICROSCOPY HANDBOOK 37, BioScientific/Springer-Verlag. Briefly, and by way of example, patient samples may be fixed in paraformaldehyde followed by methanol, blocked with a blocking solution such as horse serum, incubated with the primary antibody against polypeptide followed by a secondary antibody labeled with a fluorescent dye such as Alexa 488 and analyzed with an epifluorescent microscope.

Antibodies employed in the above-described assays may be advantageously conjugated to fluorescent dyes (e.g. Alexa488, PE), or other labels, such as quantum dots, for use in multi-parametric analyses along with other signal transduction (phospho-AKT, phospho-Erk 1/2) and/or cell marker (cytokeratin) antibodies.

In one embodiment the expression of NaPi2b in a sample from a tumor is determined immunohistochemically. In another embodiment, the expression of NaPi2b in a sample from a tumor is determined immunohistochemically (IHC) using the method described in U.S. Ser. No. 16/136,706, which is incorporated herein in its entirety by reference. In another embodiment, the expression of NaPi2b in a sample from a tumor is determined using a system such as, for example, a Leica BOND-III Fully Automated Stainer (BOND-III) system. Briefly the assay system is comprised of the following (1) a detection antibody also known as the IHC antibody (2) the IC Platform i.e. the BOND-III instrument, with an established protocol for pre-treatment, epitope retrieval and staining, as well as pre-specified control material and (3) a defined scoring method, as described in a scoring and interpretation guide.

Alternatively, the assay may include preparing RNA from the sample, optionally for use in PCR (polymerase chain reaction) or other analytical methodology. The PCR methodology is optionally, for example, RT-PCR (reverse transcription-PCR) or quantitative PCR, such as, for example, real-time RT-PCR, RNA seq and the like. Alternatively, the assaying may be conducted by use of an array, such as a microarray as known in the relevant field, such as, for example, nanostring technologies.

Patients are identified as being responsive to treatment, wherein the treatment is monitored or cancer is detected by detecting and/or measuring the expression level of NaPi2b in the tumor cells in a sample.

The detection/measurement of the expression level of NaPi2b is determined by calculating a NaPi2b score. The NaPi2b score is quantitative or semi quantitative. For example, detection is scored pathologically to arrive at a pathology score. It is contemplated that any scoring methods known in the art may be used in the methods of the invention. In particular, any histological scoring methods known in the art.

The methods for assessing the measurement results obtained by immunohistochemical staining assays include, for example, the H-score method, TPS (tumor proportion score) or PS2+ (percent score) score. The H-score (Am J Clin Pathol. 1988; 90 (3): 233-9), TPS score and PS2+ scores are determined by the following calculation formula. H-Score=((% at 0)×0)+((% at 1+)×1)+((% at 2+)×2)+((% at 3+)×3), TPS-score=(% at 1+)+(% at 2+)+(% at 3+); and PS2+score=(% at 2+)+(% at 3+); where staining intensity 0 is unstained; staining intensity 1 is weak staining; staining intensity 2 is moderate staining; and staining intensity 3 is strong staining. In some embodiments the subject having a TPS of ≥75 will be considered NaPi2b positive (high) in this assay and a TPS of <75 will be considered NaPi2b negative (low) when TPS is scored as tumor cell membrane reactivity.

In assessment by the scoring method, only cancer cell portions are used. For negative or positive controls for staining intensity, formalin-fixed paraffin-embedded cell lines or xenografts (lines whose protein expression levels are known in advance) may be employed. When there are no control specimens, a plurality of specimens are assessed simultaneously to confirm the overall distribution of staining intensity of the specimens, and then staining intensity may be set.

In addition to the scoring methods mentioned above, other scoring methods known in the art, such as, for example, the Allred method (Harvey, et al. Journal of Clinical Oncology 17, No. 5 (May 1999) 1474-1474), can also be used. Cut-off points are required to be set in each method. Allred score=score of percentage of positive cells+staining intensity score.

The disclosure also provides kits and/or methods for identifying or otherwise refining, e.g., stratifying, a patient population suitable for therapeutic administration of a NaPi2b-targeted antibody-drug conjugates disclosed herein by identifying the NaPi2b score of the subject prior to treatment with a NaPi2b-targeted antibody-drug conjugate disclosed herein. In some embodiments, the test cell population is derived from fresh, unfrozen tissue from a biopsy sample. In some embodiments, the test cell population is derived from a primary or metastatic site. In some embodiments, the test cell population is derived from a frozen tissue from a biopsy or surgical sample or ascetic fluid or pleural fluid. In some embodiments, the test cell population is derived from a fixed tissue (e.g., formalin fixation) from a biopsy or surgical sample. The IHC test measures the amount of NaPi2b receptor protein on the surface of cells in a cancer tissue sample

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “carboplatin” means a platinum co-ordination compound, diamine [1,1-cyclobutane dicarboxylato(2-)-0, 0′]. It is available commercially in various forms, such as, for example, a lyophilized powder or a pre-concentrate aqueous solution.

As used herein “platinum-sensitive recurrent disease” refers to a subject having achieved either a partial or complete response to 4 or more cycles in their penultimate platinum-containing regimen and progression of their disease >6 months after completion of the last dose of platinum containing therapy in the penultimate regimen.

As used herein “platinum-sensitive cancer” refers to a cancer that responds to treatment with anticancer drugs that contain the metal platinum, such as cisplatin and carboplatin. Cancers that respond to treatment but then come back after a certain period may also be considered platinum sensitive. For example, ovarian cancer that comes back 6 or more months after platinum-based treatment is considered platinum sensitive. Knowing whether cancer is platinum sensitive may help plan further treatment.

As used herein “recurrent disease” refers to a subject having disease progression following partial or complete response to one or more therapeutics. In some embodiments, the recurrence can be local to the original site of disease (i.e. one or more ovaries) or at a distal or metastatic location.

As used herein the term “Area under the Curve” or “AUC” defines the dose of carboplatin administered to a subject. In some aspects, the AUC is defined as the area under the plasma concentration/time curve. In some aspects, the AUC is defined as area under the free carboplatin plasma concentration versus time curve. In some aspects, the AUC is expressed as mg/ml/min. One of skill in the art would understand that AUC is a standard method and description of dosing carboplatin in a subject. As such, the skilled artisan can calculate the amount of carboplatin necessary to achieve a given AUC in a subject. See Calvert et al. Carboplatin dosage: prospective evaluation of a simple formula based on renal function. Journal of Clinical Oncology, 1989, 7(11): 1748-56, the contents of which are incorporated here in its entirety.

As used herein, the terms “NaPi2b” (also known as sodium-dependent phosphate transport protein 2B, SLC34A2, NaPiIIb, Npt2, Na(+)-dependent phosphate cotransporter 2B; sodium/phosphate cotransporter 2B; Na(+)/Pi cotransporter 2B; NaPi3b; solute carrier family 34 member 2), when used herein, refers to human NaPi2b (e.g., GenBank Accession No. 095436.3) and includes any variants, isoforms and species homologs of NaPi2b which are naturally expressed by cells, including tumor cells, or are expressed on cells transfected with the NaPi2b gene. These terms are synonymous and may be used interchangeably.

As used herein, the term “NaPi2b antibody” or “anti-NaPi2b antibody” is an antibody which binds specifically to the extracellular region of SLC34A2.

When used herein in the context of two or more antibodies, the term “competes with” or “cross-competes with” indicates that the two or more antibodies compete for binding to NaPi2b, e.g., compete for NaPi2b binding in any art-recognized assay. An antibody “blocks” or “cross-blocks” one or more other antibodies from binding to NaPi2b if the antibody competes with the one or more other antibodies 25% or more, with 25%-74% representing “partial block” and 75%-100% representing “full block”, as determined using any art-recognized assay. For some pairs of antibodies, competition or blocking in any art-recognized assay is only observed when one antibody is coated on the plate and the other is used to compete, and not vice versa. Unless otherwise defined or negated by context, the terms “competes with”, “cross-competes with”, “blocks” or “cross-blocks” when used herein is also intended to cover such pairs of antibodies

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal and chimeric antibodies.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. mAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is ≤1 μM; e.g., ≤100 nM, preferably ≤10 nM and more preferably ≤1 nM.

The term “polypeptide” is used herein as a generic term to refer to native protein, fragments, or analogs of a polypeptide sequence. Hence, native protein fragments, and analogs are species of the polypeptide genus. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Green, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, 0-phosphoserine, N-acetylserine, N-formylmethionine, 3-methylhistidine, 5-hydroxylysine, α-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH-(cis and trans), —COCH₂—, CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The use of the articles “a”, “an”, and “the” in both the following description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. For example, a polymeric scaffold of a certain formula includes all the monomer units shown in the formula and may also include additional monomer units not shown in the formula. Additionally, whenever “comprising” or another open-ended term is used in an embodiment, it is to be understood that the same embodiment can be more narrowly claimed using the intermediate term “consisting essentially of” or the closed term “consisting of.”

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “x being an integer between 1 and 6” and “x being an integer of 1 to 6” both mean “x being 1, 2, 3, 4, 5, or 6”, i.e., the terms “between X and Y” and “range from X to Y, are inclusive of X and Y and the integers there between.

All methods described herein can be performed in any suitable order unless otherwise indicated herein or otherwise clearly contradicted by context. The use of any and all examples, or exemplary language (e.g., “such as”) provided herein, is intended merely to better illustrate the invention and is not to be construed as a limitation on the scope of the claims unless explicitly otherwise claimed. No language in the specification is to be construed as indicating that any non-claimed element is essential to what is claimed.

“Polymeric Carrier or scaffold”: The term polymeric carrier or scaffold, as used herein, refers to a polymer or a modified polymer, which is suitable for covalently attaching to or can be covalently attached to one or more drug molecules with a designated linker and/or one or more PBRMs with a designated linker.

“Physiological conditions”: The phrase “physiological conditions”, as used herein, relates to the range of chemical (e.g., pH, ionic strength) and biochemical (e.g., enzyme concentrations) conditions likely to be encountered in the extracellular fluids of living tissues. For most normal tissues, the physiological pH ranges from about 7.0 to 7.4. Circulating blood plasma and normal interstitial liquid represent typical examples of normal physiological conditions.

“Drug”: As used herein, the term “drug” refers to a compound which is biologically active and provides a desired physiological effect following administration to a subject in need thereof (e.g., an active pharmaceutical ingredient).

“Cytotoxic”: As used herein the term “cytotoxic” means toxic to cells or a selected cell population (e.g., cancer cells). The toxic effect may result in cell death and/or lysis. In certain instances, the toxic effect may be a sublethal destructive effect on the cell, e.g., slowing or arresting cell growth. In order to achieve a cytotoxic effect, the drug or prodrug may be selected from a group consisting of a DNA damaging agent, a microtubule disrupting agent, or a cytotoxic protein or polypeptide, amongst others.

“PHF” refers to poly(1-hydroxymethylethylene hydroxymethyl-formal).

As used herein, the terms “polymer unit”, “monomeric unit”, “monomer”, “monomer unit”, “unit” all refer to a repeatable structural unit in a polymer.

As used herein, “molecular weight” or “MW” of a polymer or polymeric carrier/scaffold or polymer conjugates refers to the weight average molecular weight of the unmodified polymer unless otherwise specified.

As used herein, “dosing regimen” or “dosage regimen” refers to the amount of agent, for example, the composition containing an NaPi2b-targeted polymer antibody-drug conjugate, administered, and the frequency of administration. The dosing regimen is a function of the disease or condition to be treated, and thus can vary.

As used herein, “frequency” of administration refers to the time between successive administrations of treatment. For example, frequency can be days, weeks or months. For example, frequency can be more than once weekly, for example, twice a week, three times a week, four times a week, five times a week, six times a week or daily. Frequency also can be one, two, three or four weeks. The particular frequency is a function of the particular disease or condition treated. Generally, frequency is more than once weekly, and generally is twice weekly.

As used herein, a “cycle of administration” refers to the repeated schedule of the dosing regimen of administration of the enzyme and/or a second agent that is repeated over successive administrations. For example, an exemplary cycle of administration is a 28-day cycle with administration twice weekly for three weeks, followed by one-week of discontinued dosing. A preferred cycle of administration is a 21-day cycle with administration once every 21 days (i.e., 3 weeks) or a 28-day cycle with administration once every 28 days (i.e., 4 weeks)

As used herein, when referencing dosage based on mg/kg of the subject, an average human subject is considered to have a mass of about 70 kg-75 kg, such as 70 kg and a BSA of 1.73 m².

As used herein, amelioration of the symptoms of a particular disease or disorder by a treatment, such as by administration of a pharmaceutical composition or other therapeutic, refers to any lessening, whether permanent or temporary, lasting or transient, of the symptoms or, adverse effects of a condition, such as, for example, reduction of adverse effects associated with or that occur upon administration of an NaPi2b-targeted polymer antibody-drug conjugate.

As used herein, when referencing dosage based on “body surface area” (BSA; m²) is the measured or calculated surface area of a human body. For many clinical purposes BSA is a better indicator of metabolic mass than body weight because it is less affected by abnormal adipose mass. Various calculations have been published to arrive at the BSA without direct measurement. In the following formulae, BSA is in m², W is mass in kg, and H is height in cm. The most widely used is the Du Bois, Du Bois formula: BSA=0.007184×W^{0.425}×H^{0.725}. Other methods of determining BSA include for example, the Mosteller, Haycock, Gehan and George, Boyd, Fujimoto, Takahira, Shuter and Aslani or Schlich formulas.

As used herein, “treating” or “treat” describes the management and care of a patient for the purpose of combating a disease, condition, or disorder and includes the administration of a conjugate of the disclosure, or a pharmaceutical composition thereof in combination with an immunomodulatory therapy, e.g., an immuno-oncology agent such as an immune checkpoint inhibitor, to alleviate the symptoms or complications of a disease, condition or disorder, or to eliminate the disease, condition or disorder.

As used herein, “prevention” or “prophylaxis” refers to reduction in the risk of developing a disease or condition, or reduction or elimination of the onset of the symptoms or complications of the disease, condition, or disorder.

The term “effective amount” or “sufficient amount”, as it refers to an active agent, refers to the amount necessary to elicit the desired biological response. As used herein, a “therapeutically effective amount” or a “therapeutically effective dose” refers to an amount or quantity of an agent, compound, material, or composition containing a compound that is at least sufficient to produce a detectable therapeutic effect. The effect can be detected by any assay method known in the art. The precise effective amount for a subject will depend upon the subject's body weight, size, and health; the nature and extent of the condition; and the therapeutic selected for administration.

A “subject” includes a mammal. The mammal can be e.g., any mammal, e.g., a human, primate, bird, mouse, rat, fowl, dog, cat, cow, horse, goat, camel, sheep, or a pig. Preferably, the mammal is a human.

As used herein, “unit dose form” or “unit dosage form” refers to physically discrete units suitable for human and animal subjects and packaged individually as is known in the art.

As used herein, a single dosage formulation refers to a formulation as a single dose.

As used herein, “temporal proximity” refers to that administration of one therapeutic agent (e.g., a NaPi2b-targeted polymer antibody-drug conjugate disclosed herein) occurs within a time period before or after the administration of another therapeutic agent (e.g., an immune checkpoint inhibitor or carboplatin disclosed herein), such that the therapeutic effect of the one therapeutic agent overlaps with the therapeutic effect of the other therapeutic agent. In some embodiments, the therapeutic effect of the one therapeutic agent completely overlaps with the therapeutic effect of the another therapeutic agent. In some embodiments, “temporal proximity” means that administration of one therapeutic agent occurs within a time period before or after the administration of another therapeutic agent, such that there is a synergistic effect between the one therapeutic agent and the another therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

As used herein a “kit” refers to a combination of components, such as a combination of the compositions herein and another item for a purpose including, but not limited to, reconstitution, activation and instruments/devices for delivery, administration, diagnosis and assessment of a biological activity or property. Kits optionally include instructions of use. In some aspects, kits of the disclosure comprise an XMT-1535-targeted antibody polymer-drug conjugate in a sufficient amount to provide an infusion dose of between 20 mg/m² to 36 mg/m² to a subject. In some aspects, kits of the disclosure further comprise carboplatin in a sufficient amount to provide an infusion dose at a target AUC of about 5. The present disclosure is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present disclosure is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

### OTHER EMBODIMENTS

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples are illustrative and are not intended to be limiting and it will be readily understood by one of skill in the art that other reagents or methods may be utilized.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.

| Abbreviation | Full Term |
|---|---|
| ADA | Anti-drug antibodies |
| ADC | Antibody drug conjugate |
| ADL | Activities of daily living |
| AE | Adverse Event |
| AECI | Adverse Event of Clinical Interest |
| AF-HPA | Auristatin F-hydroxypropyl amide |
| ALK | Anaplastic lymphoma kinase (marker) |
| ALT | Alanine transaminase or alanine aminotransferase, or serum |
| (SGPT) | glutamate-pyruvate transaminase |
| ALP | Alkaline phosphatase |
| ANC | Absolute neutrophil count |
| AST | Aspartate transaminase or aspartate aminotransferase, or serum |
| (SGOT) | glutamic oxaloacetic transaminase |
| AUC | Area under the Curve |
| AUC_{0-Last} | Area under the curve from time 0 to the last measurable |
| | concentration. |
| BSA | Body surface area |
| Cₘₐₓ | Concentration at maximum level |
| C_{trough} | Lowest concentration of a dose just before the next dose |
| CTCAE | Common Toxicity Criteria for Adverse Events |
| DCR | Disease control rate |
| DES | Dose escalation |
| DL | Dose Level |
| DLT | Dose limiting toxicity |
| DOR | Duration of response |
| ECHO | Echocardiogram |
| eCRF | Electronic case report form |
| EXP | Expansion |
| HGSOC | High-grade, serous ovarian cancer |
| HNSTD | Highest non-severely toxic dose |
| IHC | Immunohi stochemi stry |
| IRR | Infusion-related reaction |
| mAb | Monoclonal antibody |
| MedDRA | Medical Dictionary for Regulatory Activities |
| MTD | Maximum tolerated dose |
| MUGA | Multi gated acquisition scan |
| Nab | Neutralizing antibody |
| NaPi2b | Sodium-Phosphate Transport Protein 2B or some other alias |
| NCI | National Cancer Institute |
| ORR | Objective response rate (CR + PR) |
| OS | Overall survival |
| PARPi | poly ADP ribose polymerase inhibitors |
| PD-1/PD-L1 | Programmed death-1/ Programmed death ligand-1 |
| PFS | Progression-free survival |
| PK | Pharmacokinetics |
| PR | Partial response |
| RP2D | Recommended Phase 2 Dose |
| RECIST | Response Evaluation Criteria in Solid Tumors |
| TRAE | Treatment-related Adverse Event |
| SAE | Serious Adverse Event |
| SD | Stable disease |
| SRC | Safety Review Committee |
| SRM | Safety Review Meeting |
| tₘₐₓ | Time to maximum concentration |
| t½ | Half life |
| ULN | Upper limit of normal |
| Vₛₛ | Apparent volume of distribution at steady state |
| VS | Vital signs |

### General Information

XMT-1536 was prepared as described in U.S. patent Ser. No. 10/947,317(B2).

AF-HPA was prepared as described in U.S. Pat. No. 8,808,679(B2)

CDRs were identified by the Kabat numbering scheme.

### Example 1: Assessment of Safety and Tolerability of the Combination of XMT-1536 and

Carboplatin

### Study Design

The study presented herein is an open label, multi-center Phase 1 study of XMT-1536 administered as an intravenous infusion once every 4 weeks i.e. 28-days in combination with carboplatin in participants with recurrent, platinum-sensitive high-grade serous ovarian cancer (HGSOC, including fallopian tube and primary peritoneal cancer). The trial consists of dose escalation (DES) and expansion (EXP) portions.

In the DES, the MTD will be defined as the highest dose of XMT-1536 in combination with carboplatin at a target AUC of 5 avoiding unacceptable protocol-specific dose-limiting toxicities defined by the protocol-specific dose-limiting toxicity criteria.

In the EXP portion of the trial, participants will initiate treatment at the MTD for the combination (from the DES) to determine the RP2D.

In both portions of the study, participants will be treated with up to 6 cycles of XMT-1536 in combination with carboplatin. Thereafter XMT-1536 is administered as maintenance monotherapy. Treatment is continued until disease progression, unacceptable toxicity, voluntary withdrawal or death.

The Bayesian optimal interval (BOIN) design will be used to determine the MTD in the DES portion of the study. A maximum of 18 participants will be enrolled in the DES phase, dosing participants in cohorts of size 3. Three dose levels of XMT-1536, specifically 20 mg/m², 30 mg/m², and 36 mg/m² with a BSA cap as described herein, in combination with carboplatin with an AUC of 5, will be evaluated. In the EXP portion of the trial, approximately 30 participants will be enrolled and initiate treatment at the MTD determined in the DES portion of the study. The regimen would be considered feasible if at least 60% of participants complete at least 4 cycles of the carboplatin-XMT-1536 combination without discontinuing treatment earlier for reasons other than disease progression.

In both portions of the study, participants will be treated with up to 6 cycles of XMT-1536 in combination with carboplatin followed by maintenance XMT-1536 monotherapy until disease progression, unacceptable toxicity, or voluntary discontinuation.

### Objectives and Endpoints for the DES and EXP Portions of the Study

The primary and secondary objectives and endpoints of the DES and EXP are provided in Table land Table 2 for the DES and EXP, respectively.

TABLE 1
Dose Escalation (DES) Objectives and Endpoints

| | |
|---|---|
| Objective(s) | Endpoint(s) |

Primary

| | |
|---|---|
| Determine the MTD of a once every 4-week | MTD for XMT-1536 with carboplatin at a |
| administration of XMT-1536 in combination | target AUC of 5, based on a Cycle 1 DLT |
| with carboplatin | target rate of 33% |

Secondary

| | |
|---|---|
| Assess the safety and tolerability of XMT- | Frequency and grade of adverse events based |
| 1536 in combination with carboplatin | on Common Terminology Criteria for |
| | Adverse Events (CTCAE) Version 5.0 |
| Assess PK of XMT-1536, its release product, | Cₘₐₓ, C_{trough}, tₘₐₓ, AUC, t½, Cl, and Vₛₛ |
| and selected metabolites | |
| Assess PK of carboplatin and selected | Cₘₐₓ, C_{trough}, tₘₐₓ, AUC, t½ |
| metabolites | |
| Assess the immunogenicity of XMT-1536 | Serum samples for analysis of XMT-1536 |
| when administered in combination with other | anti-drug and neutralizing antibody levels |
| agent(s) | |
| To assess preliminary anti-neoplastic activity | Objective response rate (ORR) by RECIST |
| of XMT-1536 in combination with | 1.1 |
| carboplatin | Duration of response (DOR) |
| | Disease control rate (DCR) |
| | Progression-free survival (PFS) by RECIST |
| | 1.1 |
| | Overall survival (OS) |
| To assess the association of tumor expression | Potential NaPi2b protein or RNA levels of |
| of NaPi2b and objective tumor response | NaPi2b transcript or other genes related to |
| | cancer measured in tumor samples |
| | Blood-based biomarkers, which may include |
| | serum cytokines, circulating immune cells, |
| | and circulating tumor cells |

TABLE 2
Dose Expansion (EXP) Objectives and Endpoints

| | |
|---|---|
| Objective(s) | Endpoint(s) |

Primary

| | |
|---|---|
| Assess the feasibility of the XMT-1536- | ≥60% of participants completing at least 4 |
| carboplatin combination initiated at MTD | cycles of the carboplatin-XMT-1536 |
| | combination without discontinuing treatment |
| | earlier for reasons other than disease |
| | progression. |

Secondary

| | |
|---|---|
| Assess the safety and tolerability of XMT- | Frequency and grade of adverse events based |
| 1536 in combination with carboplatin | onCTCAE v5.0 |
| Assess PK of XMT-1536, its release product, | Cₘₐₓ, C_{trough}, tₘₐₓ, AUC, t½, Cl, and Vₛₛ |
| and selected metabolites | |
| Assess PK of Carboplatin, its release product, | Cₘₐₓ, C_{trough}, tₘₐₓ, AUC, t½ |
| and selected metabolites | |
| Assess preliminary anti-neoplastic activity of | ORR by RECIST 1.1 |
| XMT-1536 in combination with carboplatin | DOR |
| | DCR |
| | PFS by RECIST 1.1 |
| | OS |
| Assess the association of tumor expression of | Potential NaPi2b protein or RNA levels of |
| NaPi2b and objective tumor response | NaPi2b transcript or other genes related to |
| | cancer measured in tumor samples |
| | Blood-based biomarkers, which may include |
| | serum cytokines, circulating immune cells, |
| | and circulating tumor cells |

### Number of Participants

Using the Bayesian Optimal Interval (BOIN) design to determine the MTD among the 3 dose levels to be evaluated in this study, a maximum sample size of 18 participants is planned in DES, with participants treated in cohorts of size typically of at least 3. In general, 6 patients for each dose level considered should be sufficient to estimate the MTD accurately.

Approximately 30 participants will be enrolled and dosed in the EXP phase. With a sample size of 30, if it is observed that 18 participants are able to tolerate at least 4 cycles of therapy, then the 80% confidence that the proportion of participants who are able to meet this feasibility criteria is between 40.6% and 77.3% based on the 2-sided 80% exact confidence interval.

Adjustments to enrollment may occur based on emergent data and Sponsor's Decision in collaboration with the Safety Review Committee (SRC).

### Eligibility

To be eligible for enrollment in this study, all participants must fulfill all the inclusion criteria and none of the exclusion criteria as defined below.

### Diagnosis and Main Criteria for Inclusion:

1. Participant must meet all of the following:
   a. Be at least 18 years of age, and female;
   b. Able to understand the study procedures and agree to participate in the study by providing informed consent; AND
   c. Have a histological diagnosis of metastatic or recurrent high grade serous ovarian cancer, which includes fallopian tube and primary peritoneal cancer;
2. Participant has received 1 to 2 prior lines of platinum containing chemotherapy for their ovarian cancer; non-platinum-based chemotherapy regimens are not permitted. They must have platinum-sensitive recurrent disease, defined as having achieved either a partial or complete response to 4 or more cycles in their last platinum-containing regimen and their disease progressing more than 6 months after completion of the last dose of platinum containing therapy. a. Clarifications for prior lines of therapy:
   i. Adjuvant±neoadjuvant is considered one line of therapy as long as the same regimens (e.g., platinum/taxane for 4 cycles before surgery followed by platinum/taxane for 4 cycles after surgery) are administered.
   ii. Maintenance therapy (e.g., bevacizumab, PARPi, endocrine therapy) will be considered as part of the preceding line of therapy (i.e., not counted independently) iii. Substitutions of different platinum agents or taxanes will not be counted as new lines.
   iv. Hormonal therapy (e.g., tamoxifen, letrozole) will be counted as a separate line of therapy unless it was given as maintenance.
3. Participant must have an ECOG performance status 0 or 1
4. Participant must have measurable or evaluable disease as per RECIST v1.1
5. Tumor sample must be provided, either an archival tumor tissue block or slides or, if not available, a tumor tissue block or slides from a new tumor biopsy obtained through a low-risk, medically routine procedure.
6. Participants with toxicity from prior therapy or surgical procedures must have recovered to ≤Grade 1. Participants with alopecia, stable immune-related toxicity such as hypothyroidism on hormone replacement, adrenal insufficiency treated with ≤10 mg daily prednisone (or equivalent), or chronic Grade 2 peripheral sensory neuropathy after prior taxane therapy is an exception to this criterion and may qualify for this study.
7. Participants must have cardiac left ventricular ejection fraction (LVEF)≥50% or ≥the institution's lower limit of normal as measured by either Echo or MVUGA scan
8. Participants must have adequate organ function within 14 days prior to enrollment as defined by the following criteria:

| | |
|---|---|
| Absolute neutrophil count (ANC), | ≥1500 cells/mm³ |
| not induced by granulocyte colony- | |
| stimulating factors | |
| Platelet count | ≥100,000/mm³ or ≥150,000/mm³ |
| Hemoglobin | ≥9 g/dL |
| INR, activated partial | within 1.2 times the institutional ULN, |
| thromboplastin time (aPTT), and | all in the absence of any other |
| prothrombin time (PT) | indicator of liver dysfunction. |
| | Participants on anticoagulants are |
| | allowed. |
| Estimated glomerular filtration rate | ≥45 mL/min according to the |
| (GFR). | Cockcroft-Gault formula |
| Total bilirubin | ≤ULN |
| | Note: Participants with asymptomatic |
| | elevations in unconjugated bilirubin |
| | due to Gilbert syndrome or stable |
| | chronic hemolytic anemia (e.g., |
| | hereditary spherocytosis, sickle cell |
| | disease, thalassemia intermedia) may |
| | be eligible after discussion with the |
| | Sponsor Medical Monitor. |
| Aspartate aminotransferase (AST | ≤1.5 times the institutional ULN |
| or SGOT) and alanine | |
| aminotransferase (ALT or SGPT). | |
| Alkaline phosphatase (ALP) | ≤1.5 times the institutional ULN, |
| | unless clearly attributable to a non- |
| | hepatic source, e.g., bone metastasis |
| Albumin | ≥3.0 g/dL |

9. A female participant is eligible to participate if she is not pregnant or breastfeeding, and at least one of the following conditions apply:
   Is not a woman of childbearing potential OR
   Is a woman of childbearing potential and using a contraceptive method that is highly effective (with a failure rate<1% per year), with low user dependency during the intervention period and for at least 6 months after the last dose of study treatment and agrees not to donate eggs (ova, oocytes), for the purpose of reproduction during this period. The Investigator should evaluate the effectiveness of the contraceptive method in relationship to the first dose of study treatment.

### Exclusion Criteria

1. Participant is unable or unlikely to comply with dosing schedule and study evaluations.
2. Participant has a prior hypersensitivity reaction to carboplatin requiring desensitization or discontinuation; or has known sensitivity to any of the study medications, or components thereof.
3. Participant has prior platelet or neutrophil toxicity to carboplatin-containing therapy requiring dose reduction to AUC<5 in the most recent regimen containing carboplatin.
4. Known history of CTCAE version 5.0 Grade 4 thrombocytopenia OR history of bleeding in association with any grade thrombocytopenia, unless approved by Medical Monitor.
5. Participant have had major surgery within 28 days of starting study treatment, systemic anti-cancer therapy within the lesser of 28 days or 5 half-lives of the prior therapy before starting study treatment (14 days or 5 half-lives for small molecule targeted therapy), or recent radiation therapy with unresolved toxicity or within a time window of potential toxicity (consultation with the Sponsor Medical Monitor is recommended).
6. Participant has a low-grade, clear cell, endometrioid, mucinous, carcinosarcoma, germ-cell, mixed histology, or stromal tumor.
7. Participant has received prior treatment with mirvetuximab soravtansine or another ADC containing an auristatin or maytansinoid payload.
8. Participant has untreated CNS metastases (including new and progressive brain metastases), history of leptomeningeal metastasis, or carcinomatous meningitis.
   a. Participants are eligible if CNS metastases are adequately treated and are neurologically stable for at least 2 weeks prior to enrollment.
   b. In addition, participants must be either off corticosteroids, or on a stable/decreasing dose of ≤10 mg daily prednisone (or equivalent). Anticonvulsants are allowed except for those drugs associated with liver toxicity.
9. Participant has clinical signs or symptoms of gastrointestinal obstruction and/or requirement for parenteral hydration or nutrition.
10. Participant has ascites or pleural effusion managed with therapeutic paracentesis or thoracentesis within 28 days prior to enrollment.
11. Participant has untreated, known human immunodeficiency virus (HIV), hepatitis B virus (HBV), or hepatitis C virus (HCV). In addition, negative serology is required during screening (baseline) for HBV and HCV:
   HBV: Participants with serologic evidence of chronic HBV infection should have an HBV viral load below the limit of quantification to be eligible.
   HCV: Participants with a history of HCV infection should have completed curative antiviral treatment and HCV viral load below the limit of quantification.
   HIV: Screening for HIV is not required except if mandated by local regulations or indicated based on clinical assessment
12. Participant has current severe, uncontrolled systemic disease (e.g., clinically significant cardiovascular, pulmonary, or metabolic disease) or intercurrent illness that could increase the risk of adverse events, whether or not potentially related to study treatment (in unclear cases, consultation with the Medical Monitor is recommended). Further, participants are excluded with the following characteristics:
   A marked baseline prolongation of QT/QTc interval (e.g., repeated demonstration of a QTc interval>480 milliseconds (ms) (CTCAE Grade 1) using Frederica's QT correction formula.
   A history of additional risk factors for Torsade's de Pointes (e.g., heart failure, hypokalemia, family history of Long QT Syndrome).
   The use of concomitant medications that prolong the QT/QTc interval will be reviewed first with the Sponsor Medical Monitor.
13. Participant has a history of cirrhosis, hepatic fibrosis, esophageal or gastric varices, or other clinically significant liver disease. Liver function testing beyond laboratory studies otherwise defined in the eligibility criteria, to diagnose potentially clinically significant liver disease based on risk factors such as hepatic steatosis or history of excessive alcohol intake, will be based on clinical judgment of the investigator.
14. Participants cannot receive drugs associated with hepatotoxicity concurrent with XMT-1536 administration. Participants may receive acetaminophen/paracetamol for a limited time but at a total daily dose of ≤2 g per day. Use of NSAIDs or steroids for treatment of fever is encouraged.
15. Current use of either constant or intermittent supplementary oxygen therapy.
16. History of or suspected pneumonitis or interstitial lung disease.
17. Oxygen saturation on room air<93%.
18. Has a diagnosis of additional malignancy that required treatment within 2 years prior to screening, except for adequately treated basal cell or squamous cell skin cancer, or carcinoma in situ of the breast or of the cervix.
19. Participant has clinically significant corneal disease, or history of clinically significant corneal disease within 12 months prior to enrollment.
20. Participant is unwilling to be transfused with blood components.
21. Participant is receiving concurrent anti-cancer therapy (e.g. chemotherapy, radiation therapy, biologic therapy, immunotherapy, hormonal therapy, investigational therapy).
22. Participant is using strong CYP450 3A4 inhibitors or inducers that cannot be discontinued while receiving study treatment.

### Criteria for Evaluation

Below is a listing of the criteria for the evaluation of a participant:
The MTD for XMT-1536 with carboplatin at a target AUC of 5, based on a Cycle 1 DLT target rate of 33%.
The frequency and grade of adverse events (AEs) based on Common Terminology Criteria for Adverse Events (CTCAE) Version 5.0
Pharmacokinetics of XMT-1536 (Cₘₐₓ, C_{trough}, tₘₐₓ, AUC, t_{1/2}, CL, and Vₛₛ)
Pharmacokinetics of carboplatin at AUC of 5 (Cₘₐₓ, C_{trough}, tₘₐₓ, AUC, t_{1/2})
Antineoplastic activity of XMT-1536 in combination with carboplatin (ORR, DOR, DCR, PFS, and OS)
Immunogenicity: Serum samples for analysis of antibodies (as defined at the timepoints in the Schedule of Activities).
Blood-based biomarkers, which may include serum cytokines, circulating immune cells, and circulating tumor cells, specified in the combination-specific module
Tissue-based biomarkers: may include measurement of NaPi2b protein or RNA level measurements of NaPi2b transcript or other genes related to cancer.

### Statistical Methods

### General Considerations

All analyses and outputs will be produced using SAS® version 9.4 or later. Unless otherwise specified, safety and efficacy summaries will be presented for each dose cohort, if applicable, and across all dose cohorts for the DES and EXP portions of the study. Continuous variables will be summarized using the number of observations (n), mean, standard deviation (SD), median, minimum, and maximum.

Categorical variables will be summarized using number of observations (n), frequency, and percentages of participants.

All relevant participant data will be included in listings. All participants enrolled in the study will be included in data listings. All by-visit summaries will use the nominal visit date. Unscheduled visits will not be summarized but will be included in the listings.

The handling of missing data will be specified in the statistical analysis plan along with the methods used for reporting the endpoints.

### Safety

Adverse events (AEs) will be coded using the Medical Dictionary for Regulatory Activities (MedDRA) for purposes of summarization. All AEs occurring during the study will be included in by-participant data listings and tabulated by MedDRA system organ class and preferred term. Safety endpoints for AEs include the following: incidence of DLTs, treatment related adverse events (TEAEs), Adverse Events of Clinical Interest (AECI), TEAEs leading to death, serious adverse events (SAEs), and AEs leading to treatment discontinuation and to study discontinuation. Tabulations of TEAEs will also be produced by severity and by relationship to study drug. Additional safety summaries will be provided for clinical laboratory tests, vital signs, ECOG performance status, and ECGs.

### Pharmacokinetics/ADA Pharmacodynamics

Serum concentrations, PK parameters, PD parameters, and ADA/nAb data will be summarized with descriptive statistics by study phase, dose, and regimen.

### Biomarkers

The incidence/changes of biomarkers will be summarized using descriptive statistics. Comparisons of clinical activity between biomarker subpopulations may be performed.

| | |
|---|---|
| Assess the association of tumor expression of | Potential NaPi2b protein or RNA levels of |
| NaPi2b and objective tumor response | NaPi2b transcript or other genes related to |
| | cancer measured in tumor samples |
| | Blood-based biomarkers, which may include |
| | serum cytokines, circulating immune cells, |
| | and circulating tumor cells |

### Dose-Limiting Toxicity Criteria

The Dose-limiting toxicity criteria includes Adverse Event (AE) and Serious Adverse Event (SAE)

### Adverse Event (AE)

An AE is any untoward medical occurrence in a patient or clinical trial participant, temporally associated with the use of trial treatment, whether or not considered related to the trial treatment. Note: Signs and symptoms and/or abnormal laboratory test results indicating a common underlying pathology/diagnosis should be reported as a single AE.

All AEs that occur after any participant has received any part of the first dose of any study drug will be reported by the participant (or when appropriate, by a caregiver, surrogate, or the subject's legally authorized representative) during the study. The investigator and any qualified designees are responsible for detecting, documenting, and recording events that meet the definition of an AE or SAE and remain responsible for following up AEs that are serious, considered related to the study treatment or trial procedures, or that caused the participant to discontinue study treatment

### Serious Adverse Event (SAE)

A serious adverse event (SAE) is defined as any untoward medical occurrence that, at any dose, that meets one or more of the criteria listed:

a. Results in death

b. Is considered life-threatening by the investigator or the sponsor

The term ‘life-threatening’ in the definition of ‘serious’ refers to an event in which the participant was at risk of death at the time of the event. It does not refer to an event, which hypothetically might have caused death, if it were more severe.

c. Requires inpatient hospitalization or prolongation of existing hospitalization

In general, hospitalization signifies that the participant has been detained (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious. Hospitalization for elective treatment of a pre-existing condition that did not worsen from baseline is not considered an AE.

d. Results in persistent disability/incapacity

The term disability means a substantial disruption of a person's ability to conduct normal life functions. This definition is not intended to include experiences of relatively minor medical significance such as uncomplicated headache, nausea, vomiting, diarrhea, influenza, and accidental trauma (eg, sprained ankle) which may interfere with or prevent everyday life functions but do not constitute a substantial disruption.

e. Is a congenital anomaly/birth defect

f. Is considered a significant medical event by the Investigator or the sponsor

Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent one of the other outcomes listed in the above definition. These events should usually be considered serious. Examples of such events include intensive treatment in an emergency room or at home for allergic bronchospasm or convulsions that do not result in hospitalization, or development of drug dependency or drug abuse.

All SAEs that occur between signing the informed consent form and prior to receiving the first dose that are caused by protocol-mandated screening procedures will be reported, i.e., hospitalization for a biopsy. SAEs are reported regardless of relationship to study drug and must be recorded on forms provided by Contract Research Organization.

The terms “severe” and “serious” are not synonymous with regard to an AE. Severity describes the intensity of an AE. A serious AE must meet one of the categories described immediately above. A participant can experience a mild, moderate, or severe SAE. A severe AE is not an SAE unless it meets one of the criteria above.

### Adverse Event Reporting

Pre-existing medical conditions will be recorded in the database as medical history items.
Exacerbations of any pre-existing conditions that occur after the administration of any part of a dose of any study drug, regardless of relationship, will be reported as treatment-emergent AEs.
Radiographic worsening or progression of cancer will not be reported as an AE or SAE because this information will be collected as part of RECIST 1.1 measurements and secondary objectives.
All adverse events must be followed until resolution or stabilization.

Investigators will assign a severity grade to all toxicities in accord with the National Cancer Institute Common Terminology Criteria for Adverse Events, version 5.0 (NCI CTCAE). NOTE: The terms toxicity and adverse events are synonymous with regard to safety reporting.

The event-specific descriptions for Grade 1: Mild; Grade 2: Moderate; Grade 3: Severe; Grade 4: Life-threatening; or Grade 5: Death related to AE will be used to grade any AE that is listed in CTCAE version 5.0. Two examples are found in Table 3

TABLE 3
Severity Definition for AEs (Toxicities) Specified in CTCAE

| Adverse Event | 1 | 2 | 3 | 4 | 5 |
|---|---|---|---|---|---|
| Platelet count | <LLN to 75.0 × 10⁹/L | <75.0 − 50.0 × 10⁹/L | <50.0 − 25.0 × 10⁹/L | <25.0 × 10⁹/L | — |
| decreased | | | | | |

Definition: A finding based on laboratory test results that indicate a decrease in number of platelets in a blood specimen.

| | | | | | |
|---|---|---|---|---|---|
| Febrile | — | — | ANC < 1000/mm³ with a | Life-threatening | Death |
| neutropenia | | | single | consequences; | |
| | | | temperature of >38.3° C. | urgent | |
| | | | (101° F.) or a sustained | intervention | |
| | | | temperature of ≥38° C. | indicated | |
| | | | (100.4° F.) for more than | | |
| | | | one hour | | |

Definition: A disorder characterized by an ANC < 1000/mm³ and a single temperature of >38.3° C. (101° F.) or a
sustained temperature of ≥38° C. (100.4° F.) for more than one hour.

However, not all AEs are specifically listed in CTCAE. When grading an AE not listed in CTCAE, use the severity descriptions in Table 4.

TABLE 4
Severity Definitions for AEs Not Individually Specified in CTCAE

| | | Medical Intervention | Impact on Activities of Daily Living |
|---|---|---|---|
| Grade | Term | Indicated | (ADL) |
| 1 | Mild | No | None |
| | Asymptomatic or minor | | |
| 2 | Moderate | Yes | Yes |
| | Minimal symptoms | Local or non-invasive | Age-appropriate instrumental ADL, |
| | | | e.g., cooking, shopping, playing sports |
| 3 | Severe | Yes | Yes |
| | Medically significant | Hospitalization or | Disabling, limited self-care ADL |
| | | prolongation | |
| 4 | Life-threatening | Yes | Yes |
| | | Urgent intervention | Requires care from another person |
| 5 | Death | Not applicable | Not applicable |
| | Related to AE | | |

SAE Reporting

SALs will be reported immediately and not more than 24 hours after first knowledge, following the instructions in the Safety Plan. A full description of an SAL is not needed at the time of initial report. The research site will follow the participant until the SAL has resolved or is stabilized and is responsible for reporting the event to their IRB/IEC in accord with that institution's rules. The Pharmacovigilance team will be responsible for ensuring all regulatory reporting rules are met for each SAL.

### Dose Limiting Toxicity (DLT) in DES (DLT) Observation Period

The DLT observation period is the time between the initiation of Cycle 1 infusion of XMT-1536 and completion of Cycle, defined as 28 days.

### Definition of Dose Limiting Toxicity

A DLT is defined as any of the following treatment-related toxicities occurring within the DLT Observation Period.

### Non-hematological Dose Limiting Toxicity:

Any case consistent with Hy's Law: AST or ALT>3×ULN and Total bilirubin>2×ULN and No other reason for liver injury
Grade 4 increase of AST or ALT of any duration
Grade≥3 pneumonitis
Infusion-related reaction related to XMT-1536 that requires hospitalization for treatment
CTCAE (v.5) Grade≥3 non hematologic toxicity not described above and not due to disease progression or another clearly identifiable cause.

The following are not considered DLTs:
Grade 3 nausea, vomiting, constipation or diarrhea persisting for <72 hours with adequate antiemetic and other supportive care.
Grade 3 fatigue
Grade≥3 electrolyte abnormality, dehydration, hypotension, dizziness or extremity edema lasting <72 hours and that resolves spontaneously or responds to conventional medical interventions
Alopecia, pain, depression, dyspepsia of any grade
Asymptomatic Grade≥3 laboratory abnormalities not otherwise defined above (e.g. Grade 3 AST or ALT elevation or Grade 3 proteinuria)

### Hematological Dose Limiting Toxicity:

CTCAE Grade 4 neutropenia (ANC<0.5×109/l) lasting >7 days or CTCAE Grade 4 thrombocytopenia (platelets<25.0×109/l) managed with platelet transfusion or lasting >7 days
Any grade hemorrhage with CTCAE Grade≥3 thrombocytopenia (25 to <50.0×109/l)
CTCAE Grade≥3 febrile neutropenia
CTCAE Grade 4 anemia not due to underlying malignancy

### General Dose Limiting Toxicity Criteria:

Any death at least possibly related to XMT-1536 or other agent(s) (not clearly due to underlying disease or extraneous cause).

### Adverse Events of Clinical Interest

Information on adverse events of clinical interest (AECI) will be sent to the Medical Monitor soon after first observation by research site staff, e.g., within 3 business days. Real-time signal monitoring will be performed on these events and a cumulative and by-dose level summary of any AECI reports will be shared as part of each SRM. In the EXP segment of the study, quarterly Safety Review Committee (SRC) meetings will be held and the following AECIs will be included in the aggregate data package.

AECI categories for the anti-tubulin drug class and based on prior observations from this study are as follows:
Infusion-related reactions Grade 3 or greater
Ocular toxicity grade 2 or greater
Pneumonitis of any grade

### Pregnancy

Details of all pregnancies in female participants and female partners of male participants will be collected after the start of the study and until the final contact at end of study. At a minimum, monthly pregnancy tests will be completed throughout the trial for all women of childbearing potential. During periods where monthly visits are not scheduled, female participants can choose to visit the site for the test or complete the test via a remote option. If a pregnancy is reported, the investigator should inform the medical monitor within 24 hours of learning of the pregnancy. Abnormal pregnancy outcomes (eg, spontaneous abortion, fetal death, stillbirth, congenital anomalies, ectopic pregnancy) are considered SAEs.

### Assessment of Causality

The investigator is obligated to assess the relationship between trial treatment and each occurrence of each AE/SAE.
The investigator will assess the relationship as either of the following:
Related: An AE will be considered “related” to the use of the IMP if there a reasonable possibility of a relationship conveys that there are facts, evidence, and/or arguments to suggest a causal relationship, rather than a relationship cannot be ruled out.
Not Related: An AE will be considered “not related” to the use of the IMP if there is no plausible causal relationship between the IMP and the AE.
The investigator will use clinical judgment to determine the relationship.
Alternative causes, such as underlying disease(s), concomitant therapy, and other risk factors, as well as the temporal relationship of the event to trial treatment administration will be considered and investigated.
The investigator will also consult the Investigator's Brochure and/or Product Information, for marketed products, in his/her assessment.
For each AE/SAE, the investigator must document in the medical notes that he/she has reviewed the AE/SAE and has provided an assessment of causality.
There may be situations in which an SAE has occurred and the investigator has minimal information to include in the initial report to the sponsor or designee. However, it is very important that the investigator always make an assessment of causality for every event before the initial transmission of the SAE data to the sponsor or designee.
The investigator may change his/her opinion of causality in light of follow-up information and send a SAE follow-up report with the updated causality assessment.
The causality assessment is one of the criteria used when determining regulatory reporting requirements.

### Dosage and Mode of Administration

Carboplatin (combined with XMT-1536) is administered at an AUC of 5 every 28 days for up to 6 cycles. Carboplatin infusion will be administered via IV infusion prior to XMT-1536 infusion. XMT-1536 will be administered via IV infusion 30 minutes (±5 minutes) after completion of the carboplatin infusion every 28 days.

XMT-1536 is provided as a colorless to light yellow frozen liquid in 5 mL vials with a rubber stopper sealed by a gray flip-off cap. Each vial contains 2.5 mL of XMT-1536 at a concentration of 10 mg/mL of ADC, pH of 4.0 to 6.0. Vials must be stored at 20° C. (±5° C.) in a temperature-monitored freezer. Each dose will be prepared in 100 mL 0.9% normal saline total volume and administered by IV infusion over 90 minutes in all participants for Cycle 1. If well tolerated, subsequent doses can be administered over 30 min. XMT-1536 will be dosed according to BSA; calculation will occur following each institution's standard practice. When possible, the Mosteller Formula will be used to calculate BSA.

### Dose Escalation (DES)

Four dose levels of XMT-1536, specifically 20 mg/m², 30 mg/m², 36 mg/m² and 43 mg/m² with a BSA cap (Table 6), in combination with carboplatin will be evaluated.

TABLE 6
XMT-1536 Dose Levels

| Dose Level^{a} | DL1^{a} | DL2^{a} | DL3^{a} | DL4^{b} |
|---|---|---|---|---|
| Dose | 20 mg/m² | 30 mg/m² | 36 mg/m² | 43 mg/m² |

Abbreviations: DL = dose level
^{a}BSA capped at 2.2 m².
^{b}BSA capped at 1.8 m².

For this study, the toxicity tolerance interval is (0.26, 0.395) and was determined based on the following:
the target DLT rate of 33%,
the highest toxicity probability that is deemed subtherapeutic (i.e., below the MTD) such that dose escalation should be made is 0.198 (=0.6×DLT rate), and
the lowest toxicity probability that is deemed overly toxic such that dose de-escalation is required is 0.462 (=1.4×DLT rate).

The escalation and de-escalation rules are equivalent to the decision boundaries in Table 7.

TABLE 7
Dose Decision Boundaries

| Number of patients treated at a dose level | 3 | 6 | 9 |
|---|---|---|---|
| Escalate if # of DLT < | 0 | 1 | 2 |
| Remain if # of DLT = | 1 | 2 | 3 |
| De-escalate if # of DLT ≥ | 2 | 3 | 4 |
| Eliminate if # of DLT ≥ | 3 | 4 | 6 |

This process of escalation and de-escalation continues until the maximum sample size of 18 participants for the study is reached. If additional dose levels/schemes are evaluated, then the maximum sample size will be increased by 6 patients for each additional dose level/scheme evaluated. Additionally, the early stopping parameter is set at 9. Therefore, if the number of participants at the current dose level reaches 9, then the trial terminates even if the maximum sample size for the study is not yet reached.

Safety rules that override the dose escalation rules will also be applied, specifically if the observed data in the current dose indicate that there is more than a 95% chance that the current dose is above the MTD and the number of participants dosed at the current dose level is ≥3, then the current dose level and higher doses are eliminated from the trial.

At the completion of the trial, isotonic regression is used to determine an efficient statistical estimate of the MTD.

### Combination of Carboplatin and XMT-1536—Cycles 1-6

The combination of carboplatin at a target AUC of 5 and XMT-1536 is administered on day 1 of each 28-day treatment cycle for a total of up to 6 cycles. The dose of XMT-1536 is assigned per patient according to the dose level in Table 8 below, at the time of patient enrollment.

TABLE 8
Dose Levels

| | | XMT-1536 (mg/m2), with a |
|---|---|---|
| Dose Level | Carboplatin Target AUC | BSA capped |
| 1 | 5 | 20^{a} |
| 2 | 5 | 30^{a} |
| 3 | 5 | 36^{a} |
| 4 | 5 | 43^{b} |

^{a}BSA capped at 2.2 m².
^{b}BSA capped at 1.8 m².

The Dose Level is selected prior to enrollment using the model-assisted design described in Tables 6 and 7.

Both carboplatin and XMT-1536 are administered via peripheral IV or in-dwelling venous catheter (e.g. Port-a-Cath). Administration procedures will ensure that the entire scheduled dose is delivered and that no part of the scheduled dose remains in the infusion line. For XMT-1536, a drug vial number is issued and provided to the pharmacist to initiate dose preparation.

Carboplatin is administered over 30-60 minutes, after which, following 30 minutes of monitoring for infusion-related reaction (IRR), XMT-1536 is then administered. XMT-1536 will be administered over 90 min for the first infusion, if well tolerated, then subsequent infusions can be administered over 30 5 minutes. Treatment of IRRs should be according to institutional standards or the guidelines given below. Any treatment for an IRR will be recorded as a Concomitant Medication and each specific infusion reaction symptom recorded as an Adverse Event.

### XMT-1536—Cycles 7 and Beyond

XMT-1536 is administered as monotherapy on day 1 of each 28-day treatment cycle using the same methods specified above. However, patients may be treated with fewer than 6 combination cycles before transitioning to XMT-1536 monotherapy with permission of the medical monitor.

### Study Treatment for EXP

The treatment plan for EXP is identical to that for DES described above; dosed at the MTD for XMT-1536 determined in DES.

### Other Embodiments

While the invention has been described in conjunction with the detailed description thereof, the foregoing description is intended to illustrate and not limit the scope of the invention, which is defined by the scope of the appended claims. Other embodiments, advantages, and modifications are within the scope of the following claims.

1. A method of treating a platinum-sensitive recurrent high grade serous ovarian cancer in a subject in need thereof, comprising administering to the subject carboplatin at a target Area Under the Curve (AUC) of about 5 and NaPi2b-targeted antibody polymer-drug conjugate by infusion at a dose of between 20 mg/m² to 36 mg/m² on the first day of treatment and every 28-days thereafter,
wherein the NaPi2b-targeted antibody polymer-drug conjugate is: 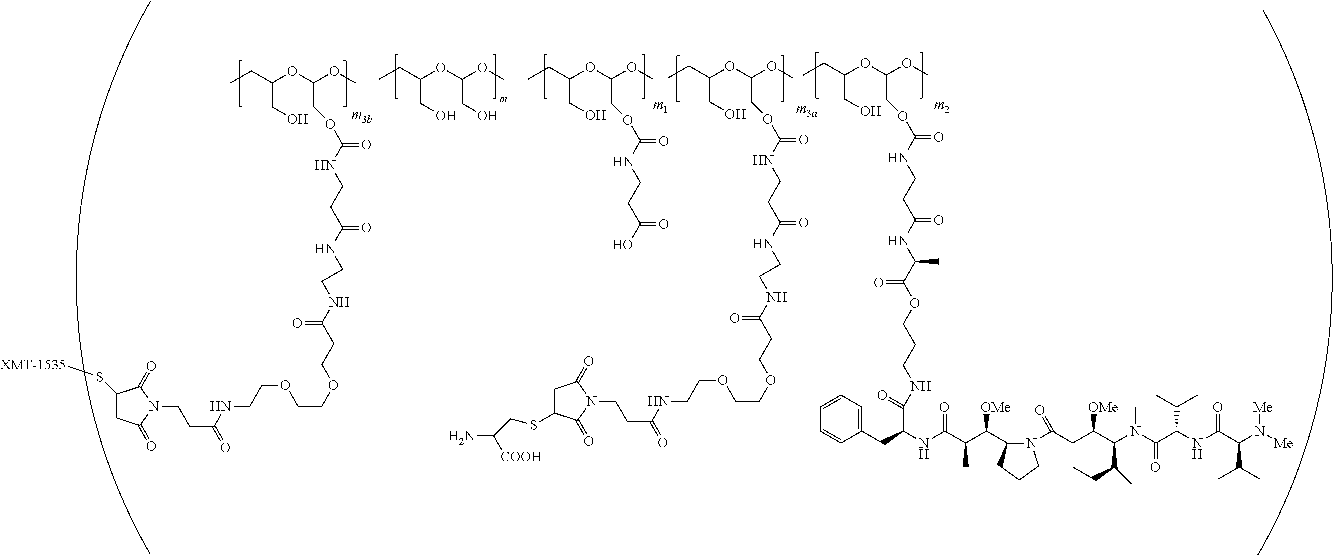
wherein:
the conjugate comprises a polymeric scaffold comprising poly(1-hydroxymethylethylene hydroxymethyl-formal) (PHF), wherein the PHF has a molecular weight ranging from 5 kDa to 10 kDa;
m is an integer from 20 to 75,
m₁ is an integer from about 5 to about 35,
m₂ is an integer from about 3 to about 10,
m₃ₐ is an integer from 0 to about 4,
m_{3b} is an integer from 1 to about 5,
the sum of m, m₁, m₂, m₃ₐ, and m_{3b} ranges from about 40 to about 75,
XMT-1535 comprises a variable light chain complementarity determining region 1 (CDRL1) comprising the amino acid sequence SASQDIGNFLN (SEQ ID NO: 8); a variable light chain complementarity determining region 2 (CDRL2) comprising the amino acid sequence YTSSLYS (SEQ ID NO: 9); a variable light chain complementarity determining region 3 (CDRL3) comprising the amino acid sequence QQYSKLPLT (SEQ ID NO: 10); a variable heavy chain complementarity determining region 1 (CDRH1) comprising the amino acid sequence GYTFTGYNIH (SEQ ID NO: 5); a variable heavy chain complementarity determining region 2 (CDRH2) comprising the amino acid sequence AIYPGNGDTSYKQKFRG (SEQ ID NO: 6); and a variable heavy chain complementarity determining region 3 (CDRH3) comprising the amino acid sequence GETARATFAY (SEQ ID NO: 7); and
wherein the ratio between the PHF and XMT-1535 is an integer from 2 to about 6.

2. The method of claim 1, wherein XMT-1535 comprises a variable heavy chain comprising the amino acid sequence of SEQ ID NO: 3 and a variable light chain comprising the amino acid sequence of SEQ ID NO: 4.

3. The method claim 1, wherein XMT-1535 comprises a heavy chain comprising the amino acid sequence of SEQ ID NO: 1 and a light chain comprising the amino acid sequence of SEQ ID NO: 2.

4. The method of claim 1, wherein the conjugate dose is 20 mg/m² is capped at BSA 2.2 m².

5. The method of claim 1, wherein the conjugate dose is 30 mg/m² and is capped at BSA 2.2 m².

6. The method of claim 1, wherein the conjugate dose is 36 mg/m² and is capped at BSA 2.2 m².

7. The method of claim 1, wherein the conjugate dose is 80 mg.

8. The method of claim 1, wherein the carboplatin is administered prior to the NaPi2b-targeted antibody polymer-drug conjugate.

9. The method of claim 1, wherein the high grade serous ovarian cancer is a recurrent high grade serous ovarian cancer.

10. The method of claim 1, wherein the high grade serous ovarian cancer is fallopian tube cancer or primary peritoneal cancer.

11. The method of claim 1, wherein the subject is administered carboplatin and the NaPi2b-targeted antibody polymer-drug conjugate on the first day of treatment and every 28-days thereafter for up to 6 cycles.

12. The method of claim 11, wherein after the subject is administered carboplatin and the NaPi2b-targeted antibody polymer-drug conjugate by infusion for up to 6 cycles, the subject is administered the NaPi2b-targeted antibody polymer-drug conjugate at a maintenance dose of 36 mg/m², 30 mg/m², or 20 mg/m².

13. The method of claim 11, wherein after the subject is administered carboplatin and the NaPi2b-targeted antibody polymer-drug conjugate by infusion for up to 6 cycles, the subject is administered the NaPi2b-targeted antibody polymer-drug conjugate at a maintenance dose of about 80 mg.

14. The method of claim 1, wherein PHF has a molecular weight ranging from about 5 kDa to about 10 kDa, m is an integer from 30 to about 35, m₁ is an integer from 8 to about 10, m₂ is an integer from 2 to about 5, m₃ₐ is an integer from 0 to about 1, m_{3b} is an integer from 1 to about 2, the sum of m₃ₐ and m_{3b} ranges from 1 and about 4, and the ratio between the PHF and XMT-1535 is about 3 to about 5.

15. The method of claim 1, wherein the ratio between m2 and XMT-1535 is about 16:1 to 10:1.

16. The method of claim 1, wherein the ratio between m2 and XMT-1535 is about 12:1 to 8:1.


SEQUENCE LISTING (15 sequences)

SEQ ID NO: 1 - protein, 449 aa, homo sapiens
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 215 aa, homo sapiens
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, homo sapiens
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSG

SEQ ID NO: 4 - protein, 108 aa, homo sapiens
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKR

SEQ ID NO: 5 - protein, 10 aa, homo sapiens
GYTFTGYNIH

SEQ ID NO: 6 - protein, 17 aa, homo sapiens
AIYPGNGDTSYKQKFRG

SEQ ID NO: 7 - protein, 10 aa, homo sapiens
GETARATFAY

SEQ ID NO: 8 - protein, 11 aa, homo sapiens
SASQDIGNFLN

SEQ ID NO: 9 - protein, 7 aa, homo sapiens
YTSSLYS

SEQ ID NO: 10 - protein, 9 aa, homo sapiens
QQYSKLPLT

SEQ ID NO: 11 - protein, 329 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 107 aa, homo sapiens [sequence omitted]

SEQ ID NO: 13 - DNA, 357 bp, homo sapiens
caagttcagctggttcagtctggcgccgaggttgtgaaacctggcgcctctgtgaagatgagctgcaaggccagcggctacaccttcaccggctacaacatccactgggtcaagcaggcccctggacagggactcgaatggatcggagccatctatcccggcaacggcgacaccagctacaagcagaagttccggggcagagccacactgaccgccgatacaagcaccagcaccgtgtacatggaactgagcagcctgagaagcgaggacagcgccgtgtactattgcgccagaggcgaaacagccagagccacctttgcctattggggccagggaaccctggtcaccgttagctct

SEQ ID NO: 14 - DNA, 321 bp, homo sapiens
gatattcagatgacacagagccccagcagcctgtctgcctctgtgggagacagagtgaccatcacctgtagcgccagccaggatatcggcaacttcctgaactggtatcagcagaaacccggcaagaccgtgaaggtgctgatctactacacctccagcctgtacagcggcgtgcccagcagattttctggcagcggctctggcaccgactacaccctgaccatatctagcctgcagcctgaggacttcgccacctactactgccagcagtacagcaagctgcccctgacatttggccagggcaccaagctggaactgaag

SEQ ID NO: 15 - protein, 690 aa, homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL